(19) United States
(12) Patent Application Publication
(10) Pub. No.: US 2020/0108089 A1
(43) Pub. Date: Apr. 9, 2020

(54) Gemcitabine Derivatives for Cancer Therapy

(71) Applicants: Suzhou Sirnaomics Biopharmaceuticals Co., Ltd., Suzhou (CN); Sirnaomics, Inc., Gaithersburg, MD (US)

(72) Inventors: Patrick Y. Lu, Potomac, MD (US); Aslam Ansari, Gaithersburg, MD (US); Parker J. Guan, Germantown, MD (US); John J. Xu, Germantown, MD (US); Vera Simonenko, Germantown, MD (US); Tom Zhong, Suzhou (CN)

(73) Assignees: Suzhou Sirnaomics Biopharmaceuticals Co., Ltd., Suzhou (CN); Sirnaomics, Inc., Gaithersburg, MD (US)

(21) Appl. No.: 16/495,294

(22) PCT Filed: Mar. 19, 2018
(86) PCT No.: PCT/US2018/023148

§ 371 Date: Sep. 18, 2019

Related U.S. Application Data
(60) Provisional application No. 62/473,441, filed on Mar. 19, 2017.

(51) Int. Cl. A61K 31/7068; A61K 31/713; C12N 15/113; A61K 31/575; A61K 47/64; A61P 35/00

(52) U.S. Cl. CPC A61K 31/7068; A61K 31/713; C12N 15/1136; C12N 15/1137; A61K 31/575; C12N 2310/113; A61P 35/00; C12N 15/1138; C12N 2310/14; C12N 2320/31; C12N 2310/141; A61K 47/6455

(57) ABSTRACT

The present invention provides pharmaceutical compositions comprising the chemotherapy drug gemcitabine (GEM) and certain derivatives, a taurocholic acid (TCA) formulation, and a Histidine-Lysine Polymer (HKP) conjugate, for enhancement of RNAi cancer therapeutics.

### CROSS-REFERENCE TO RELATED PATENT APPLICATIONS

This application claims the benefit of and priority to U.S. Provisional Patent Application No. 62/473,441, filed Mar. 19, 2017, which is incorporated herein by reference in its entirety.

### FIELD OF THE INVENTION

The invention relates to gemcitabine-based compounds, compositions, and formulations and their use as cancer therapeutics, alone or with RNA interference (RNAi) compounds.

### BACKGROUND OF THE INVENTION

### Pancreatic Cancer Treatment is an Urgent Unmet Need

Pancreatic cancer is one of the malignancies with the worst prognosis because of aggressive invasion, early metastasis, and almost complete resistance to existing chemotherapeutic agents and radiation therapy (1). In the past few years, the use of gemcitabine (2′,2′-difluorodeoxycytidine) has been shown to result in improved clinical symptoms and slightly longer overall survival in pancreatic cancer patients. Thus, gemcitabine has become the first-line treatment option for pancreatic cancer (2). However, chemoresistance to gemcitabine is increasing and has become a major cause of clinical treatment failure for pancreatic cancer. It is proposed that resistance to gemcitabine is mainly attributed to increased resistance to apoptosis (3). Consequently, new therapeutic strategies to induce apoptosis and enhance chemosensitivity to gemcitabine are urgently needed in this disease.

siRNA Cancer Therapeutics

RNA interference (RNAi) is an endogenous process of gene inhibition offering a potent means to inhibit expression of virtually any gene. The RNAi technology has become a wide accepted tool for functional genetics in cell culture and animal disease models, and thus it holds great promise for therapeutic applications. The understanding of the key roles of TGF-β1, COX-2, mTOR, EGFR, and RAF1, involved in pathways in Pancreatic Cancer growth and development, prompted us to consider use of RNA interference (RNAi) as an alternative therapeutic approach. One advantage of small interfering RNA (siRNA) drugs is the potent inhibitory effect on those gene targets, since the reduction in protein level should amplify the inhibition. Another advantage is the facility to evaluate inhibition of different members of the pathways and thus identify the most effective targets more efficiently.

miRNA Cancer Therapeutics

MicroRNAs (miRNAs) are a class of 18-24 nucleotide non-coding RNAs, whose principal function is to regulate the translation of coding mRNA transcripts. Physiologic regulation of the cellular transcriptome by miRNAs plays a critical role during development and in mature tissue homeostasis. Aberrant expression of miRNA is common in human cancers, and miRNAs can be over or under expressed in neoplastic cells compared to their normal counterparts (4, 5). The underlying basis for aberrant miRNA expression in cancer can be manifold, including genomic alterations (amplifications and deletions), epigenetic mechanisms, or altered transcription factor regulation (5, 6). In many instances, the coding mRNA targets of aberrant miRNAs have been elucidated, and include transcripts whose protein products regulate critical cell growth, cell death, and metastatic machineries in cancer cells (4-9).

One miRNA molecule, miR-132, has been characterized to facilitate pathological angiogenesis by down regulating p120 RasGAP, a molecular brake for Ras. Targeting miR-132 with a synthetic antagomir oligo decreased angiogenesis and tumor burden in multiple tumor models. A recent work demonstrated that miR-132 is increased in pancreatic cancer and targeted the retinoblastoma tumor suppressor (3). Another miRNA, miR-155, exhibited an elevated expression in pancreatic tumors that is associated with poor survival (4). miR-155 appears to be a biomarker of early pancreatic neoplasia, and it warrants further evaluation as a pancreatic cancer biomarker (5). The role of miR-155 has been linked to repression of p53-mediated tumor suppression (6), and it has also been indicated as involving tumorigenic activities with other tumor types (7, 8). Recently, we have packaged antagomir-132 and antagomir-155, the modified RNA oligos, with Histidine-Lysine co-polymer (HKP) into nanoparticles and tested this dual-targeted inhibitor with a viral-induced mouse model of Herpetic Stromal Keratitis (10). The profound anti-angiogenesis effect of this dual-targeting miR-132 and miR-155 approach was observed with all treated mice.

Development of oligo nucleotide therapeutics relies on efficient delivery of the active pharmaceutical ingredient, such as antagomir oligos. We will test HKP for systemic delivery of the dual-targeted antagomirs-132/155 using mouse xenograft tumor models with human BxPC-3 or Panc-1 pancreatic tumor cells (11-14).

### A Chemo-Drug and RNAi Delivery System

Many chemotherapy applications have been used for treatment of pancreatic cancer and other cancer types. The chemo-resistance and chemo-drug toxicity concerns have limited its therapeutic potential. This invention combines the strengths of RNAi therapeutics and Gemcitabine, a chemo-drug already in clinical applications, using a Gemcitabine derivative for delivery of siRNA or miRNA.

Gemcitabine (2′-difluorodeoxycytidine) is a nucleoside analogue that exhibits antitumor activity. Gemcitabine exhibits cell phase specificity, primarily killing cells undergoing DNA synthesis (S-phase) and blocking the progression of cells through the G1/S-phase boundary. Gemcitabine is metabolized intracellularly by nucleoside kinases to the active diphosphate (dFdCDP) and triphosphate (dFdCTP) nucleosides. The cytotoxic effect of gemcitabine is attributed to a combination of two actions of the diphosphate and the triphosphate nucleosides, which leads to inhibition of DNA synthesis. First, gemcitabine diphosphate inhibits ribonucleotide reductase, which is responsible for catalyzing the reactions that generate the deoxynucleoside triphosphates for DNA synthesis. Inhibition of this enzyme by the diphosphate nucleoside causes a reduction in the concentrations of deoxynucleotides, including dCTP. Second, gemcitabine triphosphate competes with dCTP for incorporation into DNA. The reduction in the intracellular concentration of dCTP (by the action of the diphosphate) enhances the incorporation of gemcitabine triphosphate into DNA (self-potentiation). After the gemcitabine nucleotide is incorporated into DNA, only one additional nucleotide is added to the growing DNA strands. After this addition, there is inhibition of further DNA synthesis. DNA polymerase epsilon is unable to remove the gemcitabine nucleotide and repair the growing DNA strands (masked chain termination). In CEM T lymphoblastoid cells, gemcitabine induces internucleosomal DNA fragmentation, one of the characteristics of programmed cell death.

Gemcitabine was first described in U.S. Pat. No. 4,808,614, incorporated herein by reference in its entirety, as an antiviral compound. The anti-tumor properties of gemcitabine were later described in U.S. Pat. No. 5,464,826, incorporated herein by reference in its entirety. The formulation teachings of U.S. Pat. Nos. 4,808,614 and 5,464,826, incorporated herein by reference in their entirety, provide that the compounds claimed therein can be administered parenterally, and that a dried powder, which is then reconstituted in an aqueous solution, is preferred. Currently, gemcitabine is marketed as a freeze-dried parenteral that is then reconstituted by the administrating personnel prior to administration by injection or infusion.

The term “gemcitabine” as used herein means gemcitabine free base and certain gemcitabine derivatives. Those derivatives are chemical structure related with minor modification and have the same prodrug properties.

The U.S. Food and Drug Administration (FDA) first approved gemcitabine hydrochloride for sale in the United States in 1996 as an injectable formulation under the tradename GEMZAR® (Eli Lilly & Co., Indianapolis, Ind.). The clinical formulation is supplied in a sterile form for intravenous use only. Vials of GEMZAR® contain either 200 mg or 1 g of gemcitabine HCl (expressed as free base) formulated with mannitol (200 mg or 1 g, respectively) and sodium acetate (12.5 mg or 62.5 mg, respectively) as a sterile lyophilized powder. Hydrochloric acid and/or sodium hydroxide may have been added for pH adjustment.

Gemcitabine demonstrates dose-dependent synergistic activity with cisplatin in vitro. No effect of cisplatin on gemcitabine triphosphate accumulation or DNA double-strand breaks was observed. In vivo, gemcitabine showed activity in combination with cisplatin against the LX-1 and CALU-6 human lung xenografts, but minimal activity was seen with the NCI-H460 or NCI-H520 xenografts. Gemcitabine was synergistic with cisplatin in the Lewis lung murine xenograft. Sequential exposure to gemcitabine 4 hours before cisplatin produced the greatest interaction.

GEMZAR® is indicated as in combination with cisplatin for the first-line treatment of patients with locally advanced (Stage IIIA or metastatic (Stage IV) NSCLC.

GEMZAR® is also available as first-line treatment of the treatment of locally advanced (nonresectable Stage II or Stage III) or metastatic pancreatic cancer (Stage IV) in patients. However, the toxicity of gemcitabine limits the dosage of drug that can be administered to patients. Gemcitabine HCL also has very short half-life in patients (half-life for short infusions ranged from 32 to 94 minutes). The half-life and volume of distribution depends on age, gender and duration for infusion. Moreover, the development of multidrug resistance in cells exposed to gemcitabine can limit its effectiveness. Consequently, formulations of gemcitabine are needed that sufficiently prolong half-life of gemcitabine and maximize its therapeutic efficacy for example, by minimizing the multidrug resistance of treated cells and limiting its toxicity.

### BRIEF DESCRIPTION OF THE FIGURES

FIG. 1. The schematic illustration of the concept of using an anti-cancer chemo-drug as a RNAi therapeutic delivery vehicle. Gemcitabine (GEM) is chemically conjugated with a Histidine-Lysine Polymer (HKP) to form a new chemical entity GEM-HKP. This GEM-HKP is able to carry an siRNA which is specific to a tumor target gene with a nanoparticle formulation. This due anti-cancer activities through Gemcitabine and oncogene inhibitory siRNA may represent a novel cancer therapeutic approach.

FIG. 2. Comparison of silencing potencies between 25mer and 21mer siRNA duplexes. The most potent 25 mer and 21mer siRNA were selected first from each set of 6 duplexes. Than comparison was carried out with two tumor cell lines expressing human VEGF protein (DLD-1, colon carcinoma and MBA-MD-435, breast carcinoma) using in vitro transfection with Lipo2000 (Invitrogen, CA) followed by RT-PCR analyses. At either 0.3 μg or 2.0 μg doses, 25mer siRNA demonstrated stronger inhibitory activity than 21mer siRNA, especially at 2.0 μg.

FIG. 3. Selection of potent siRNA targeting mTOR. (A) The lower panel illustrates selection of eight 25 mer siRNA duplexes with control siRNA were transfected into human MDA-MB-231 cells and mouse CT26 cells. 24 hr later, mRNA was collected and subject to Q-RT-PCR with the standard control gene target Rigs15. The panel demonstrates selection of potent mTOR-siRNA using Q-RT-PCR following transfections of human MDA-MB-231 cells and mouse CT26 cells.

FIG. 4. Knockdown of miR-132 by antagomir-132 nanoparticles in mouse eyes. Antagomir-132 treatment regimen resulted in peak miR-132 knockdown in the corneas (A) (Pooled n=6 mice/group). One way ANOVA with Bonferroni's post hoc test was used to calculate the level of significance. P≤0.05 (*). Six corneas were collected and pooled for analysis by QPCR or WB. (B) Antagomir-132 and scrambled sequences were injected in HSV infected mice subconjunctively and the quantification of RasGAP mRNA from corneas isolated from different groups was carried out (n=6 mice/group). The level of significance was determined by student's t test (unpaired). ***P≤0.001.

FIG. 5. Potent anti-angiogenesis activity was observed with dual-targeted antagomirs-132/155. WT mice and miR-155 KO mice were infected with HSV-1 RE in one eye. The anti-angiogenesis effect was measured with a score of angiogenesis on day 12 and 15 p.i. The dual-targeted antagomirs-132/155 exhibits most potent activity among all three groups on day 15 p.i. The level of significance was determined by student's t test (unpaired). P≤0.001 (***); P≤0.01 (**); P≤0.05 (*). Error bars represent means±SE. These experiments were repeated twice.

FIG. 6. Schematic illustration of Gemcitabine and Taurocholic Acid combination Chemical Structures of Gemcitabine and Taurocholic Acid, which can be formulated into GEM-TCA. This novel formulation will have a dual function, serving as both an anti-cancer drug and RNAi delivery vehicle.

FIG. 7 Cytotoxicity Comparison between GEMZAR® and GEM-TCA. 1×10³ HeLa cells were seeded on the wells of 96-well plate in 150 ul of EMEM/10% FBS. The next day, the medium was supplemented with 0.1 nM-100 uM GEMZAR® or GEM-TCA diluted in the same medium. At 72 h post chemical exposure cytotoxicity was assessed with Cell Titer-Glo Luminescent cell viability assay (Promega). Values derived from untreated cells (Blank) were set as 100%. All values represent the mean of ±S.D. of four replicates for each dilution.

FIG. 8. Cytotoxicity Comparison between GEMZAR® and GEM-TCA 2×10³ Panc-1 and HepG2 cells were seeded on the wells of 96-well plate in 150 ul of EMEM/10% FBS. The next day, the medium was supplemented with 0.1 nM-100 uM GEMZAR® or GEM-TCA diluted in the same medium. At 72 h post chemical exposure cytotoxicity was assessed with Cell Titer-Glo Luminescent cell viability assay (Promega). Values derived form untreated cells (Blank) were set as 100%. All values represent the mean of ±S.D. of four replicates for each dilution.

FIG. 9. Effect of forward transfection with mTOR_{siRNA} on chemosensitivity of Panc-1 cells to GEM-TCA 5×10³ Panc-1 cells were seeded on the wells of 96-well plate in 100 ul of DMEM/10% FBS. The next day cells were transfected with siRNA/Lipofectamine 2000 complexes accordingly to the manufactures' recommendations. In 5-6 h. medium was changed. The next day various concentrations of GEM-TCA were applied to the transfected cells. At 72 h post chemical exposure cytotoxicity was assessed with Cell Titer-Glo Luminescent cell viability assay (Promega). Values derived from untreated cells (Blank) were set as 100%. All values represent the mean of ±S.D. of four replicates for each dilution. * different from cells transfected with control, not-targeting siRNA (p<0.05, Student's t test)

FIG. 10. Effect of forward transfection with TGF-β1_{siRNA} and mTOR_{siRNA} chemosensitivity of Panc-1 cells to GEM-TCA The next day, the medium was supplemented with 3.9 nM-1000 nM GEM-TCA diluted in the same medium. At 48 h post chemical exposure cytotoxicity was assessed with Cell Titer-Glo Luminescent cell viability assay (Promega). Values derived from untreated cells (Blank) were set as 100%. All values represent the mean of ±S.D. of four replicates for each dilution. Paired sample two-tailed Student's t-test was used to determine significance.

FIG. 11. Particle Size Measurement for GEM-TCA/siRNA Nanoparticle Formulation. Measurements of nanoparticle sizes at various ratios of GEM-TCA to siRNA payload in comparison with GEMZAR®/siRNA formulation.

FIG. 12. Particle Zeta Potential Measurement for GEM-TCA/siRNA Nanoparticle Formulation. Measurements of nanoparticle Zeta potential and sizes at various ratios of GEM-TCA to siRNA payload in comparison with GEMZAR®/siRNA formulation.

FIG. 13. The Chemical Structure of HKP (H3K4b) for conjugation to Gemcitabine as a novel anti-cancer approach

FIG. 14. The Chemical Conjugation Route of Gemcitabine with HKP. This is a general concept to make a covalent bond between Gembitabine and HKP through a (please add what is missing here.). It has a special character that the lone pair of electron at nitrogen relocated into carbonyl, finally form C═N double bond, and a hydroxyl. Actually, the amide will be acid catalyzed hydrolysis into carboxyl. That means the only “C-terminal” of HKP turn back carboxyl group at acid condition. It becomes the unique breakout that we can take advantage of for modification. We can modify HKP through the C-terminal carboxyl group.

FIG. 15. The EDC-NHS Chemistry for Conjugation of Gemcitabine and HKP The advantage of using EDC-NHS chemistry:

1. EDC-NHS reaction occurs most effectively at acid condition.
2. HKP will generate carboxyl group under acid condition.
3. EDC-NHS reaction prefer —NH₂ rather than —NH₃⁺.
—NH₂ of Gemcitabine outstands from interfering amines of HKP at acid condition due to the low pKa value (˜2.8), which make Gemcitabine conjugate with HKP instead of HKP self-conjugation.

FIG. 16. The Wavelength of HKP and GEM-HKP Comparing with HKP, the Gemcitabine is much smaller molecule (40× smaller), as shown in the proposed reaction mechanism, one molecule Gemcitabine added on HKP will not retard the HKP peak position much. And also, although Gemcitabine has absorbance at ˜205 nm as well, if under equal-molar level, its absorbance is negligible comparing to HKP. What's more, we didn't find any other strong peaks at longer or shorter time point (from 0 to 60 min). Based on the HPLC and UV results, we can make the conclusions below: The proposed HKP-Gemcitabine (GEM-HKP) compound is synthesized successfully. The new compound has one gemcitabine binding with one HKP. No significant side product was observed.

FIG. 17. Measurements of HKP, Gemcitabine, HKP/Gemcitabine Mixture, and GEM-HKP Conjugate through size exclusion at different UV absorbance HKP and Gemcitabine as shown in different molecular weights: HKP (9.6 kD) and Gemcitabine (236D). With size exclusion column measurements, we found that HKP and Gemcitabline came out at different time points. The HKP peak appeared at ˜19 min, whereas the Gemcitabine peak appeared at ˜5 min. Gemcitabine has no absorbance at ˜19 min at all. However, when GEM-HKP was measured, this single compound exhibits the absorbance at both 205 nm and 272 nm, and shows two peaks at ˜19 min together.

FIG. 18. Measurement of GEM-HKP Physiochemical properties The particle sizes and Zeta potential of nanoparticle formation when GEM-HKP aqueous solution and siRNA aqueous solution mixed together at a 4:1 ratio. The scrambled siRNA was used with GEM-HKP to form nanoparticles and the original HKP was used as positive control under the same condition. The size and Zeta potential of the nanoparticles were measured using Brookhaven 90Plus Nanosizer: the average particles sizes of GEM-HKP is 78.4 nm with Zeta potentials of 25 mV. The nanoparticle of GEM-HKP/siRNA has similar Zeta potential with that of HKP/siRNA, but smaller particle size.

FIG. 19 GEM-HKP Delivers siRNA into Panc-1 Cells We then used AF488 siRNA (scrambled siRNA modified with Fluorescent AF488) as reporter to form nanoparticles together with GEM-HKP to evaluate their capability for in vitro siRNA transfection. HKP-siRNA nanoparticle was used as control. Our new compound, GEM-HKP, has an ability to deliver siRNA into the cells with the similar efficiency with HKP. Panc-1 cell line was used as model for this evaluation.

FIG. 20. GEM-HKP Cytotoxic Activity for Killing the Tumor Cells. Non-specific AF488 labeled siRNA was transfected Panc-1 cells with HKP or GEM-HKP at a ratio of carrier:siRNA as 4.5:1. Twenty-four hours post-transfection, medium containing siRNA and transfection agent or drug alone were replaced with fresh medium. At 48 hours and 72 hour post transfection, the images of cell growth were taken for evaluation of cell killing. Although the cell killing activity was not very clear at 24 hour post transfection, the GEM-HKP carried siRNA nanoparticle has demonstrated potent cell killing activity. The result suggests that GEM-HKP is able to preserve the properties of siRNA delivery (HKP function) and tumor cell killing (Gemcitabine function). Therefore, GEM-HKP may represent a novel anti-tumor agent also to delivery therapeutic siRNA drugs.

FIG. 21. Dosage-Dependent Cytotoxicities of Gemcitabine and GEM-HKP conjugate in the Panc-1 cell culture, at 72 hrs post treatments After exposures of the Panc-1 cells to Gemcitabine only, GEM-HKP conjugate, the cytotoxicities of each treatment was assessed with a “Cell Titer-Glo Luminescent cell viability assay” (Promega). Values derived from untreated cells (Blank) were set as 100%. All values represent the mean of ±S.D. of four replicates for each dilution. In this study, the HKP concentration at each point equals to its concentration in the Gem/HKP. As shown in the Figure, the cytotoxicity of GEM-HKP is comparable to that of Gemcitabine, while HKP has shown no cytotoxicity.

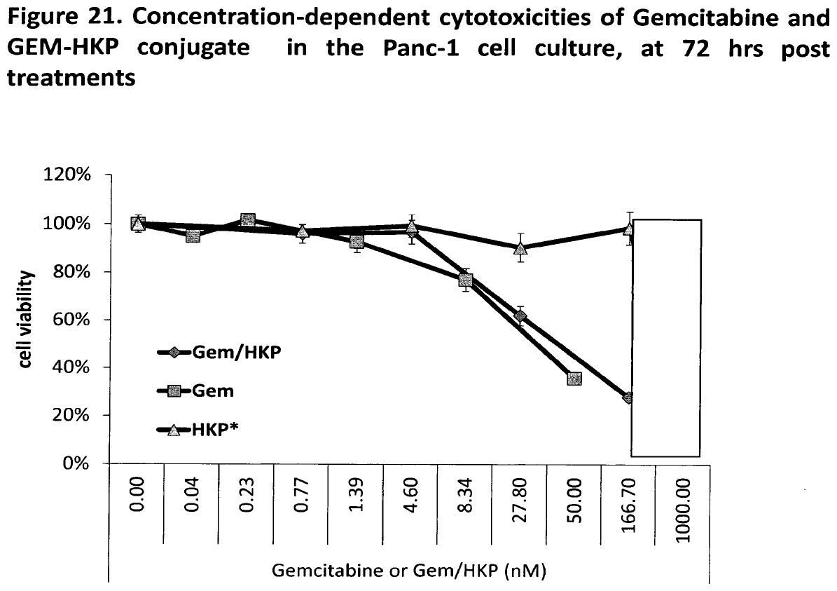

FIG. 22. Tumor Inhibition Test with A549 (Lung Cancer) Cell Xenograft Mouse Model. MOD is the tumor model group without treatment. GEM is the tumor model group treated with GemZar. GEM-TCA is the tumor model group treated with Gemcitabine-Taulichoric Acid formulation. Cohort group N=6. GemZar and GEM-TAC were used with the same dosage.

FIG. 23. Tumor Inhibition Test with PANC-1 (human pancreatic Cancer) Cell Xenograft Mouse Model. MOD is the tumor model group without treatment. GEM is the tumor model group treated with GemZar. GEM-TAC is the tumor model group treated with Gemcitabine-Taulichoric Acid formulation. Cohort group N=5. GemZar and GEM-TAC were used with the same dosage.

FIG. 24. Tumor Inhibition Test with PANC-1 (human pancreatic Cancer) Cell Xenograft Mouse Model. MOD is the tumor model group without treatment. GEM is the tumor model group treated with GemZar. GEM-TAC is the tumor model group treated with Gemcitabine-Taulichoric Acid formulation. Cohort group N=8. GemZar and GEM-TAC were used with the same dosage. There is significant difference between the therapeutic benefits of GemZar and GEM-TAC.

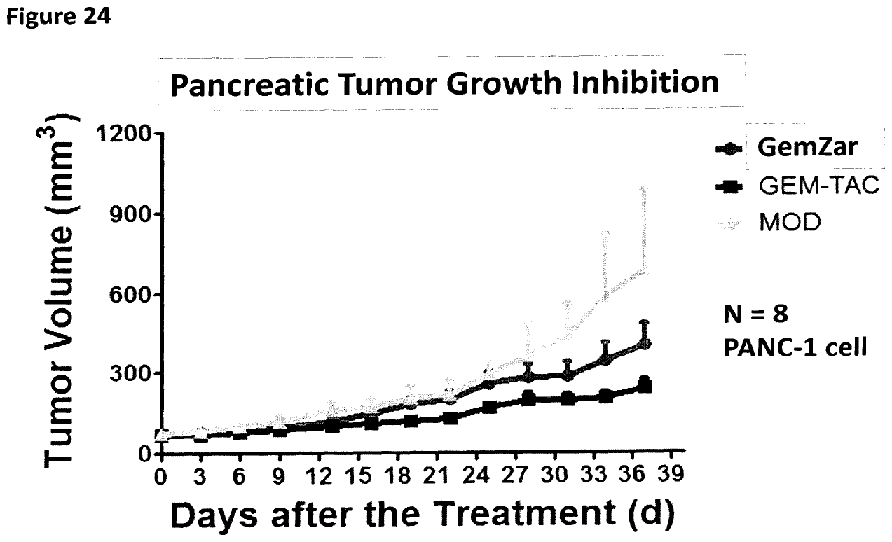

FIG. 25. Tumor Inhibition Test with PANC-1 (human pancreatic Cancer) Cell Xenograft Mouse Model by total tumor weight on day 37 post treatment. MOD is the tumor model group without treatment. GEM is the tumor model group treated with GemZar. GEM-TAC is the tumor model group treated with Gemcitabine-Taulichoric Acid formulation. Cohort group N=8. GemZar and GEM-TAC were used with the same dosage. There is significant difference between the therapeutic benefits of GemZar and GEM-TAC.

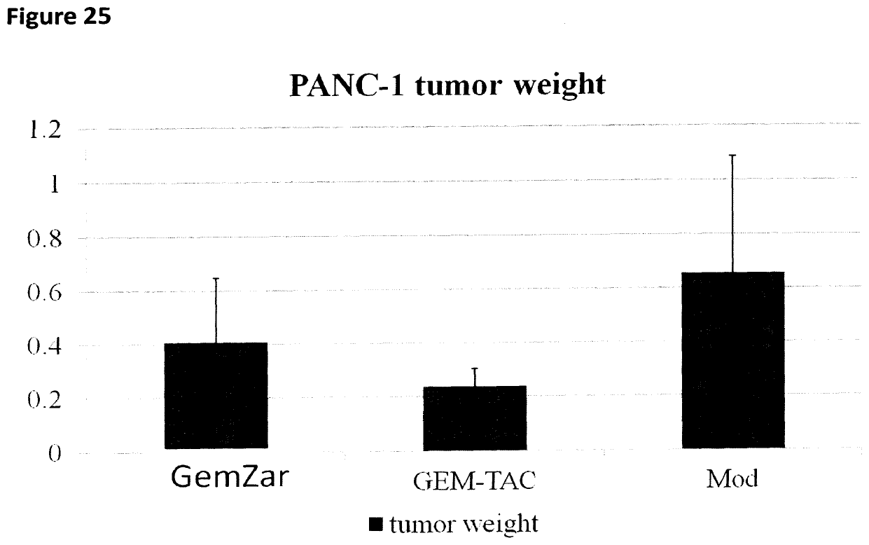

FIG. 26. Tumor Inhibition Test with LoVo (human Colon Cancer) Cell Xenograft Mouse Model by Intratumor Injection. MOD is the tumor model group without treatment. STP302 is a miRNA therapeutic candidate with mir150/HKP formulation. GEM-TAC is the tumor model group treated with Gemcitabine-Taulichoric Acid formulation. Cohort group N=6GEM-TAC+STP302 combination resulted in better efficacy than their individual use.

FIG. 27. Tumor Inhibition Test with LoVo (human Colon Cancer) Cell Xenograft Mouse Model by Intratumor Injection and harvested at day 16 post injection. MOD is the tumor model group without treatment. STP302 is a miRNA therapeutic candidate with mir150/HKP formulation. GEM-TAC is the tumor model group treated with Gemcitabine-Taulichoric Acid formulation. Cohort group N=6. GEM-TAC+STP302 combination resulted in better efficacy than their individual use.

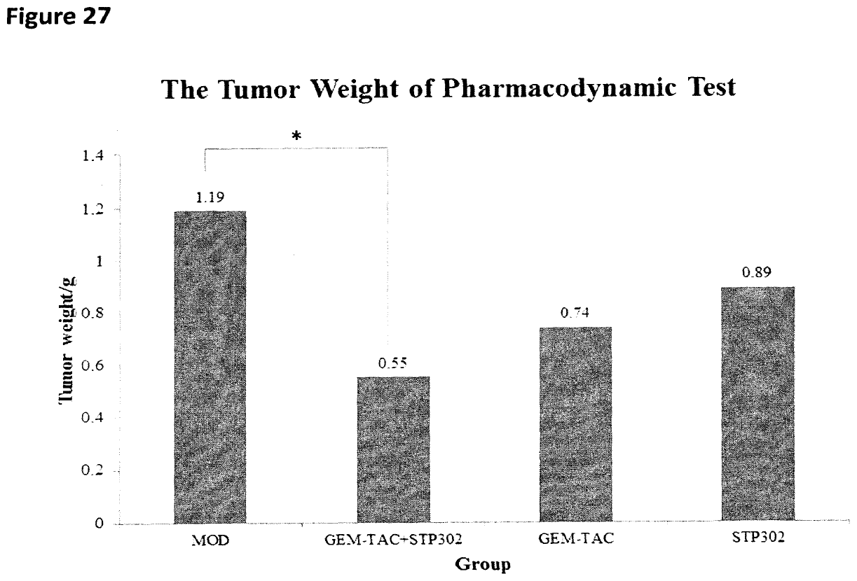

FIG. 28. Tumor Inhibition Test with LoVo (human Colon Cancer) Cell Xenograft Mouse Model by Intratumor Injection. MOD is the tumor model group without treatment. GEM is the tumor model group treated with GemZar. GEM-TAC is the tumor model group treated with Gemcitabine-Taulichoric Acid formulation. Cohort group N=8. GemZar and GEM-TAC were used with the same dosage. There is significant difference between the therapeutic benefits of GemZar and GEM-TAC.

FIG. 29. Tumor Inhibition Test with LoVo (human Colon Cancer) Cell Xenograft Mouse Model by Intratumor Injection, measured at day 18 post injection. MOD is the tumor model group without treatment. GEM is the tumor model group treated with GemZar. GEM-TAC is the tumor model group treated with Gemcitabine-Taulichoric Acid formulation. Cohort group N=8. GemZar and GEM-TAC were used with the same dosage. There is significant difference between the therapeutic benefits of GemZar and GEM-TAC.

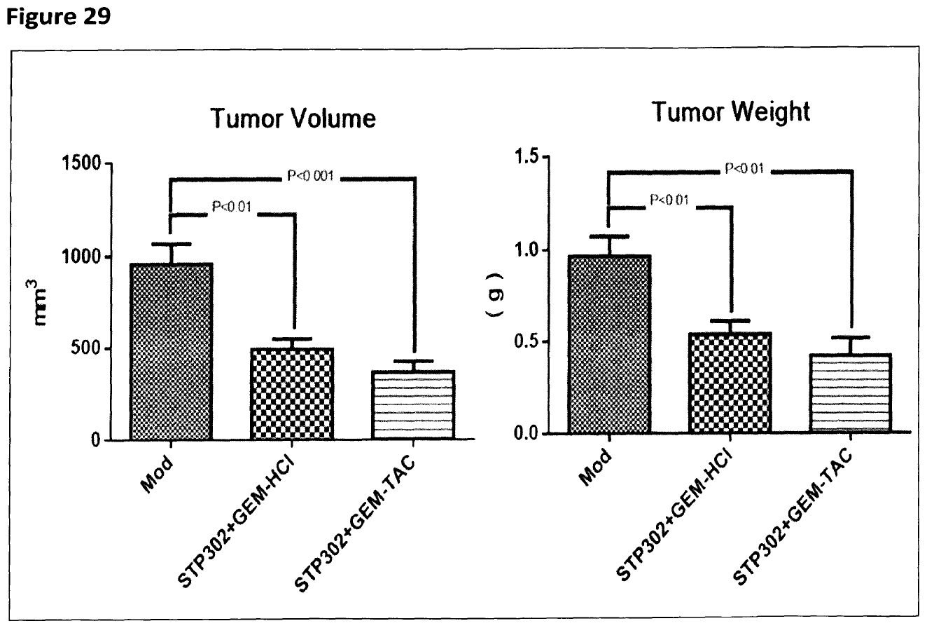

FIG. 30. Positive siRNA Sequences against Human PDL-1 were Identified. Multiple siRNA sequences were screened for inhibition of PDL-1 gene expression using human cervical cancer cell line, Caski cell culture. Positive siRNA sequences were marked with stars.

FIG. 31. Additional Positive siRNA Sequences against Human PDL-1 were Identified. Multiple siRNA sequences were screened for inhibition of PDL-1 gene expression using human cervical cancer cell line, Caski cell culture. Positive siRNA sequences were marked with star.

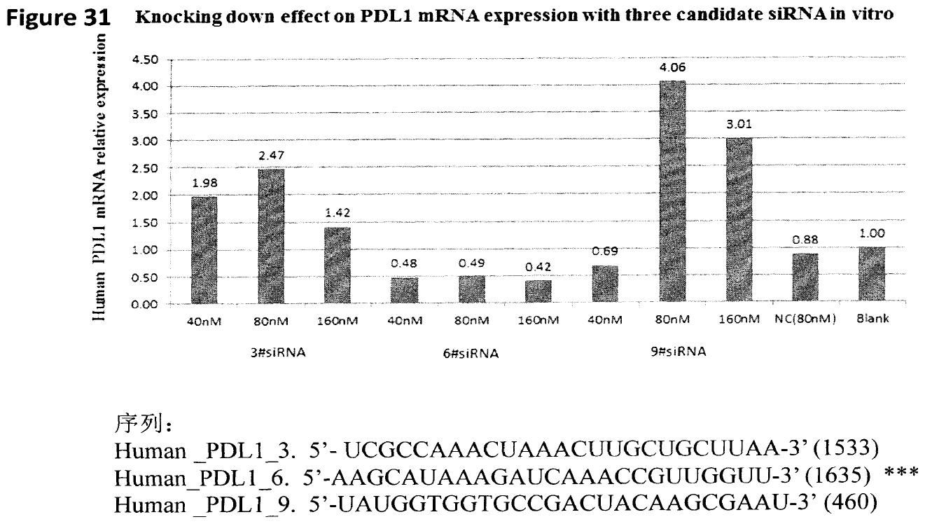

FIG. 32. Positive siRNA Sequences against Human PDL-2 were Identified. Multiple siRNA sequences were screened for inhibition of PDL-2 gene expression using human cervical cancer cell line, Caski cell culture. Positive siRNA sequences were marked with stars.

FIG. 33. Positive siRNA Sequences against Human PDL-2 were Identified. Multiple siRNA sequences were screened for inhibition of PDL-2 gene expression using human cervical cancer cell line, Caski cell culture. Positive siRNA sequences were marked with stars.

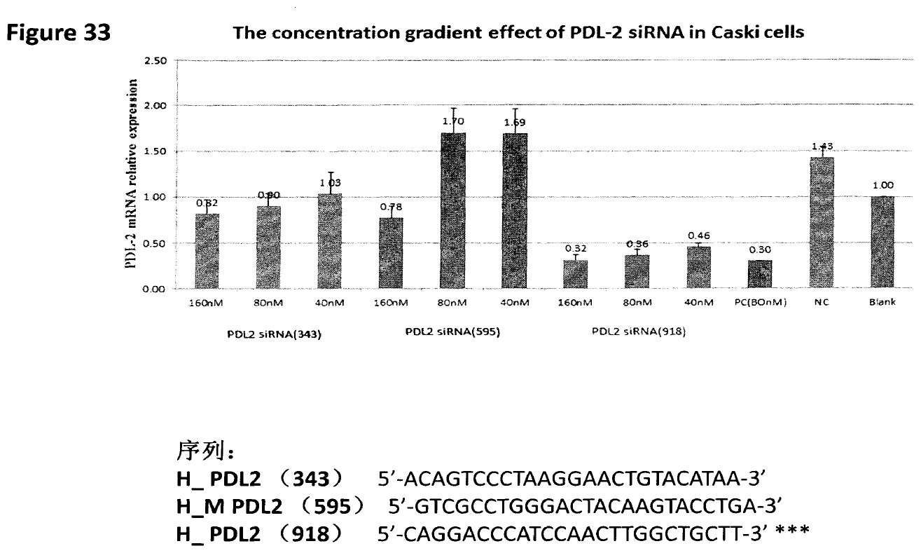

### DESCRIPTION OF THE INVENTION

The present invention provides pharmaceutical compositions comprising the chemo drug gemcitabine (GEM) and certain derivatives, a taurocholic acid (TCA or TAC) formulation, and a Histidine-Lysine Polymer (HKP) conjugate, for cancer therapy and for enhancement of RNAi cancer therapeutics. A first embodiment comprises a GEM and TCA formulation (GEM-TCA), an anti-cancer therapeutic composition for treatment of various types of cancers, such as the cancers in mammals and more particularly in humans. A second embodiment comprises a GEM and HKP conjugate (GEM-HKP) for treatment of various types of cancers. A third embodiment comprises a therapeutic composition comprising GEM-TCA for efficient siRNA or miRNA delivery or both. A fourth embodiment comprises a therapeutic composition comprising GEM-HKP for efficient siRNA or miRNA delivery or both. A fifth embodiment comprises methods of using of those pharmaceutical compounds, formulations, and compositions for various therapeutic conditions, including cancer therapeutics.

As used herein, the singular forms “a,” “an,” and “the” refer to one or more, unless the context clearly indicates otherwise.

The invention includes a pharmaceutical composition comprising a gemcitabine derivative and an RNAi trigger. In one aspect of this embodiment, the gemcitabine derivative comprises a gemcitabine molecule in electrostatic attraction with a taurocholic acid molecule. In another aspect of this embodiment, gemcitabine is combined with a taurocholic acid composition comprising deoxycholic acid with taurine. In still another aspect, the gemcitabine and the taurocholic acid are in a mole ratio of about 0.0:0.1 to 1.0:2.0. In another aspect of this embodiment, the gemcitabine derivative comprises a chemical conjugate comprising a gemcitabine molecule and a Histidine-Lysine Polymer. The gemcitabine may be in the form of the free base. In still another aspect, the composition further comprises a second RNAi trigger different from the first.

Histidine-Lysine Polymers are described in U.S. Pat. Nos. 7,070,807 B2, 7,163,695 B2, and 7,772,201 B2, which are incorporated herein by reference in their entireties. In one aspect of this embodiment, the HKP comprises the structure (R)K(R)-K(R)-(R)K(X), where R=KHHHKHHHKHHHKHHHK, K=lysine, and H=histidine.

The RNAi trigger is any molecule that activates an RNAi effect in a human cell or other mammalian cell. Such RNAi triggers include a small interfering RNA (siRNA) oligo, a micro RNA (miRNA) oligo, or an antagomir oligo.

As used herein, an “siRNA oligo,” an “siRNA molecule” or an “siRNA duplex” is a duplex oligonucleotide, that is a short, double-stranded polynucleotide, that interferes with the expression of a gene in a cell, after the molecule is introduced into the cell, or interferes with the expression of a viral gene. For example, it targets and binds to a complementary nucleotide sequence in a single stranded (ss) target RNA molecule. SiRNA molecules are chemically synthesized or otherwise constructed by techniques known to those skilled in the art. Such techniques are described in U.S. Pat. Nos. 5,898,031, 6,107,094, 6,506,559, 7,056,704 and in European Pat. Nos. 1214945 and 1230375, which are incorporated herein by reference in their entireties. By convention in the field, when an siRNA oligo is identified by a particular nucleotide sequence, the sequence refers to the sense strand of the duplex molecule.

One or more of the ribonucleotides comprising the molecule can be chemically modified by techniques known in the art. In addition to being modified at the level of one or more of its individual nucleotides, the backbone of the oligonucleotide can be modified. Additional modifications include the use of small molecules (e.g. sugar molecules), amino acids, peptides, cholesterol, and other large molecules for conjugation onto the siRNA molecule.

In one aspect, the siRNA molecule is a double-stranded oligonucleotide with a length of about 17 to about 27 base pairs. In one further aspect, the molecule is a double-stranded oligonucleotide with a length of 19 to 25 base pairs. In another aspect, it is a double-stranded oligonucleotide with a length of 25 base pairs. In all of these aspects, the molecule may have blunt ends at both ends, or sticky ends with overhangs at both ends (unpaired bases extending beyond the main strand), or a blunt end at one end and a sticky end at the other. In one particular aspect, it has blunt ends at both ends. In another particular aspect, the molecule has a length of 25 base pairs (25 mer) and has blunt ends at both ends.

In one aspect of this embodiment, the siRNA molecules are the molecules identified by their sense sequence in Table 1.

In another aspect of this embodiment, the siRNA oligo has specific sequence homology (preferably 100%) to mTOR gene mRNA and has an inhibitory activity to mTOR gene expression. An example of such an siRNA oligo is mTOR-siRNA:

sense,
5′-r(CACUACAAAGAACUGGAGUUCCAGA)-3′,
antisense,
5′-r(UCUGGAACUCCAGUUCUUUGUAGUG)-3′.

In still another aspect of this embodiment, the siRNA oligo has specific sequence homology (preferably 100%) to TGF-β1 gene mRNA and has an inhibitory activity to TGF-β1 gene expression. An example of such an siRNA oligo is TGF-β1-siRNA:

sense,
5′-r(CCCAAGGGCUACCAUGCCAACUUCU)-3′,
antisense,
5′-r(AGAAGUUGGCAUGGUAGCCCUUGGG)-3′.

In still another aspect of this embodiment, the siRNA oligo has specific sequence homology (preferably 100%) to COX-2 gene mRNA and has an inhibitory activity to COX-2 gene expression. An example of such an siRNA oligo is COX-2-siRNA:

sense,
5′-r(GGUCUGGUGCCUGGUCUGAUGAUGU)-3′,
antisense,
5′-r(ACAUCAUCAGACCAGGCACCAGACC)-3′.

In a further aspect of this embodiment, the miRNA oligo comprises or has homology (preferably 100%) to miR-132 (accguggcuuucgauuguuacu), miR-150 (ucucccaacccuuguaccagug), or miR-155 (uuaaugcuaaucgugauagggguu).

In still a further aspect of this embodiment, the antagomir comprises or has homology (preferably 100%) to antagomir-132 (accguggcuuucgauuguuacu), antagomir-150 (ucucccaacccuuguaccagug), or antagomir-155 (uuaaugcuaaucgugauagggguu).

In another aspect of this embodiment, the compositions are combined with a pharmaceutically acceptable carrier. Such carriers are determinable by those skilled in the art, given the teachings contained herein.

The invention also includes a pharmaceutical composition comprising a gemcitabine molecule and a taurocholic acid molecule. The gemcitabine may be in the form of the free base. In one aspect of this embodiment, the taurocholic acid comprises a deoxycholic acid with taurine. In a further aspect of this embodiment, the composition further comprises an RNA interference (RNAi) trigger as described above. A still further aspect of this embodiment, the composition comprises a second RNAi trigger different from the first. In another aspect of this embodiment, the compositions are combined with a pharmaceutically acceptable carrier. Such carriers are determinable by those skilled in the art, given the teachings contained herein.

The invention further includes a pharmaceutical composition comprising a gemcitabine molecule and a Histidine-Lysine Polymer (HKP). The gemcitabine may be in the form of the free base. In one aspect of this embodiment, the composition further comprises an RNA interference (RNAi) trigger as described above. In another aspect of this embodiment, the composition comprises a second RNAi trigger different from the first. In further aspect of this embodiment, the compositions are combined with a pharmaceutically acceptable carrier. Such carriers are determinable by those skilled in the art, given the teachings contained herein.

The compositions of the invention are useful in the treatment of cancers and other neoplastic disease in humans and other mammals.

The invention provides a method of treating cancer in a mammal or inhibiting the growth of neoplastic or tumor cells in a mammal comprising the step of administering a therapeutically effective amount of any of the compositions of the invention to the mammal. In one aspect of the invention, the neoplastic or tumor cells are pancreatic cancer cells.

The invention also provides method of inducing apoptosis of neoplastic or tumor cells in a mammal comprising the step of administering an effective amount of any of the compositions of the invention to the mammal. In one aspect of the invention, the neoplastic or tumor cells are pancreatic cancer cells.

The invention further provides a method of enhancing chemosensitivity of a mammal with cancer to GEM comprising the step of administering an effective amount of any of the compositions of the invention to the mammal. In one aspect of the invention, the cancer is pancreatic cancer.

Mammals include humans and laboratory animals, such as nonhuman primates, dogs, and rodents. In one embodiment of the invention, the mammal is a human.

The following examples illustrate certain aspects of the invention and should not be construed as limiting the scope thereof.

### Example 1. Targeted Cancer Therapeutics with Chemo-Drug Delivered siRNA

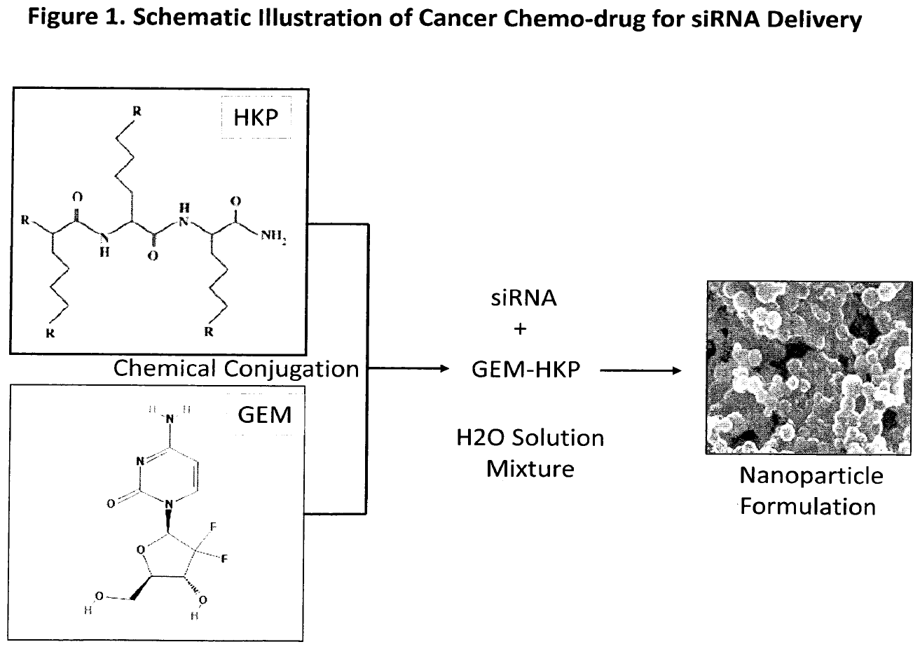

Many chemo-therapies have been used for treatment of pancreatic cancer and other types of cancers. Chemo-resistance and chemo-drug toxicity concerns limit their therapeutic potential. This invention combines the strengths of RNAi therapeutics and Gemcitabine, a chemo-drug already in clinical applications, for delivery of siRNA or miRNA. FIG. 1 illustrates a schematic process whereby Gemcitabine and the polypeptide carrier HKP can be chemically conjugated with characteristics of the two components, tumor cell killing and siRNA or miRNA delivery in vitro and in vivo. When this new compound, GEM-HKP, mixed with a mTOR specific siRNA in an aqueous solution with certain ratio, self-assembled nanoparticles will be formed with properties of mTOR-targeted siRNA therapeutics, and Gemcitabine-mediated tumor cell killing (FIG. 1).

### Example 2. 25mer Demonstrated Stronger Inhibitory Activity than 21mer

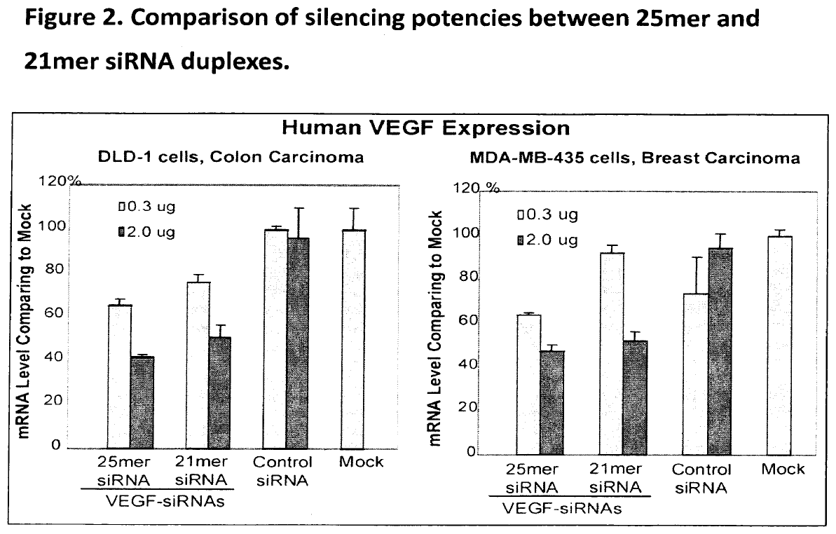

First, we found that 25mer siRNA is more potent than 21mer siRNA for target gene silencing. In one of the experiments, we compared the silencing potencies between a 25mer and 21mer siRNAs which were selected from each set of 6 duplexes. The comparison were conducted with two tumor cell lines prepressing human VEGF protein (DLD-1, human colon carcinoma and MBA-MD-435, human breast carcinoma) using in vitro transfection with Lipo2000 followed by RT-PCR analyses. As seen in FIG. 2 that the 25mer siRNA demonstrated stronger inhibitory activity than 21mer siRNA at both 0.3 ug and 2.0 ug dosages. In addition, we have demonstrated through an ocular angiogenesis mouse model that the cocktail siRNA targeting VEGF, VEGFR1 and VEGFR2 exhibited stronger anti-angiogenesis activity than the single siRNA inhibitor. Furthermore, packaging siRNA into the HKP nanoparticle provided us a systemic siRNA delivery system. The anti-tumor activity of HKP-Raf1-siRNA and HKP-EGFR-siRNA demonstrated through MBA-MD-435 xenograft tumor model strongly support our effort of using HKP to enhance the EGFR-RAF1-mTOR or VEGFR2-RAF1-mTOR siRNA cocktail therapeutic effect for treatment of Pancreatic Cancer.

### Example 3. Selection of Potent siRNA Targeting mTOR Gene Expression

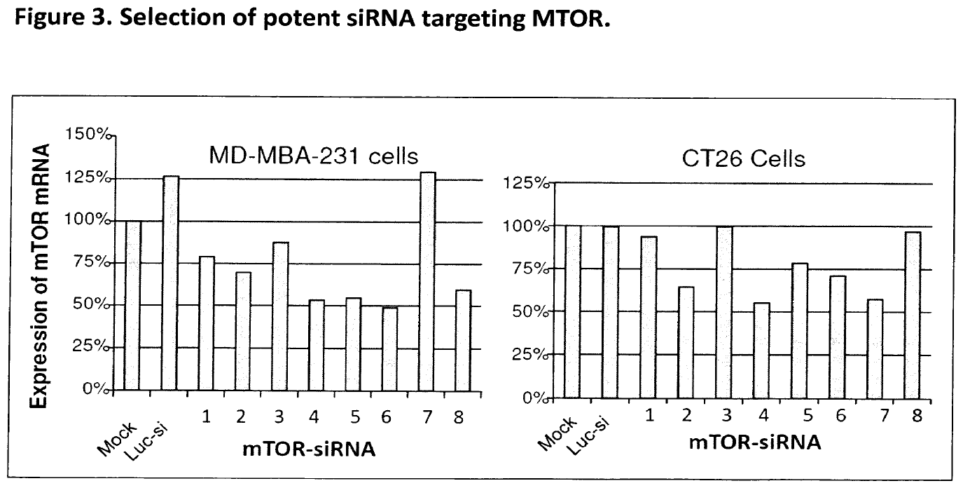

In our proof-of-concept and feasibility studies using nanoparticle-mediated siRNA cocktail for cancer treatment, we first found that the most potent siRNA duplexes targeting EGFR, VEGFR2, RAF-1 and mMTOR genes (both Human and Mouse) were identified and validated through cell culture followed by Q-RT-PCR and Western Blot analyses. For mTOR siRNA selection, we first use in silico screening selected 8 siRNA sequences for siRNA oligo synthesis. And then we transfected these siRNAs into human MDA-MB-231 cells and mouse CT26 cells. Twenty-four hours later, the total mRNA collected and subjected to qRT-PCR analysis with the standard control gene target Rigs15. From FIG. 3 we can see that the potent siRNA duplexes targeting mTOR (both human and mouse mRNAs) was selected.

### Example 4. Knockdown of miR-132 and miR-155 for Potential Anti-Cancer Therapeutics

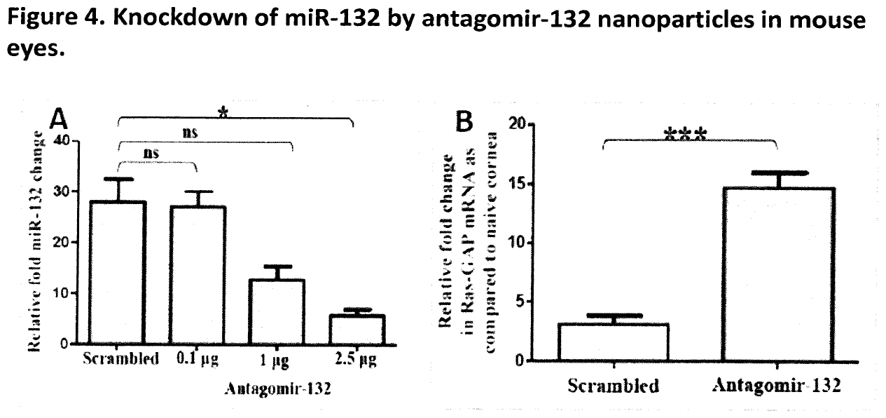

Antagomir-132 treatment regimen resulted in peak miR-132 knockdown in the corneas (A) (Pooled n=6 mice/group). One way ANOVA with Bonferroni's post hoc test was used to calculate the level of significance. P≤0.05 (*). Six corneas were collected and pooled for analysis by QPCR or WB. (B) Antagomir-132 and scrambled sequences were injected in HSV infected mice subconjunctively and the quantification of RasGAP mRNA from corneas isolated from different groups was carried out (n=6 mice/group). The level of significance was determined by student's t test (unpaired). ***P≤0.001 (FIG. 4).

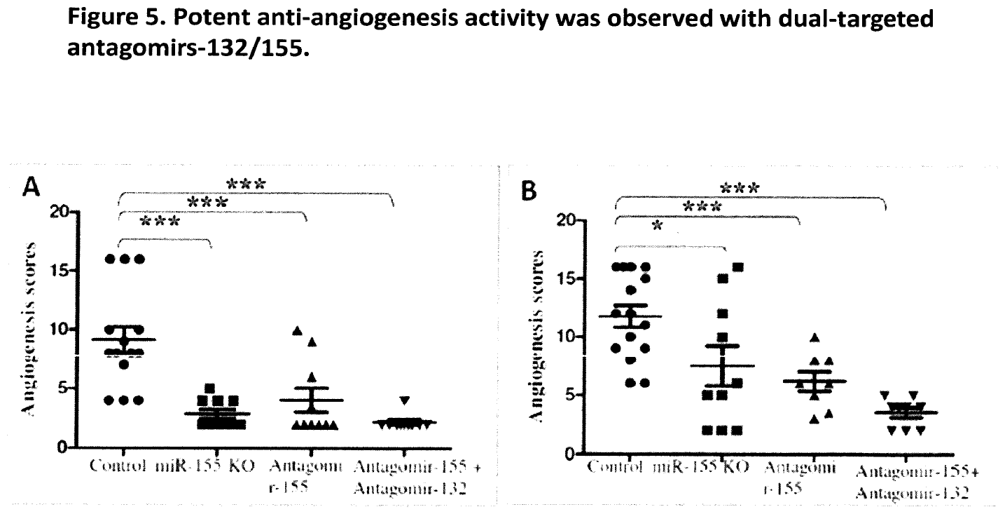

Increase of miR-155 in mouse pancreatic cancer tissue and pancreatic cancer patient plasma with observed with a correlation between the expression of target gene mRNA and miR-155 in mouse normal and pancreatic cancer tissue (PDAC), using q RT-PCR. Detection of miR-155 levels in human plasma samples from pancreatic cancer patients, non-cancer controls, and patients with other GI cancers, where pancreatic cancer versus non-cancer controls with pancreatic disease, non-cancer controls without pancreatic disease, upper GI cancer, colon cancers, and liver cancers. * p, 0.05. WT mice and miR-155 KO mice were infected with HSV-1 RE in one eye. The anti-angiogenesis effect was measured with a score of angiogenesis on day 12 and 15 p.i. The dual-targeted antagomirs-132/155 exhibits most potent activity among all three groups on day 15 p.i. The level of significance was determined by student's t test (unpaired). P≤0.001 (***); P≤0.01 (**); P≤0.05 (*). Error bars represent means±SE (FIG. 5).

### Example 5. Gemcitabine and Taurocholic Acid Combination Formulation

Gemcitabine (dFdC) is a new anticancer nucleoside that is an analog of deoxycytidine. It is a pro-drug and, once transported into the cell, must be phosphorylated by deoxycytidine kinase to an active form. Both gemcitabine diphosphate (dFdCTP) and gemcitabine triphosphate (dFdCTP) inhibit processes required for DNA synthesis. Incorporation of dFdCTP into DNA is most likely the major mechanism by which gemcitabine causes cell death. After incorporation of gemcitabine nucleotide on the end of the elongating DNA strand, one more deoxynucleotide is added and thereafter, the DNA polymerases are unable to proceed. This action (“masked termination”) apparently locks the drug into DNA as the proofreading enzymes are unable to remove gemcitabine from this position. Furthermore, the unique actions that gemcitabine metabolites exert on cellular regulatory processes serve to enhance the overall inhibitory activities on cell growth. This interaction is termed “self-potentiation” and is evidenced in very few other anticancer drugs.

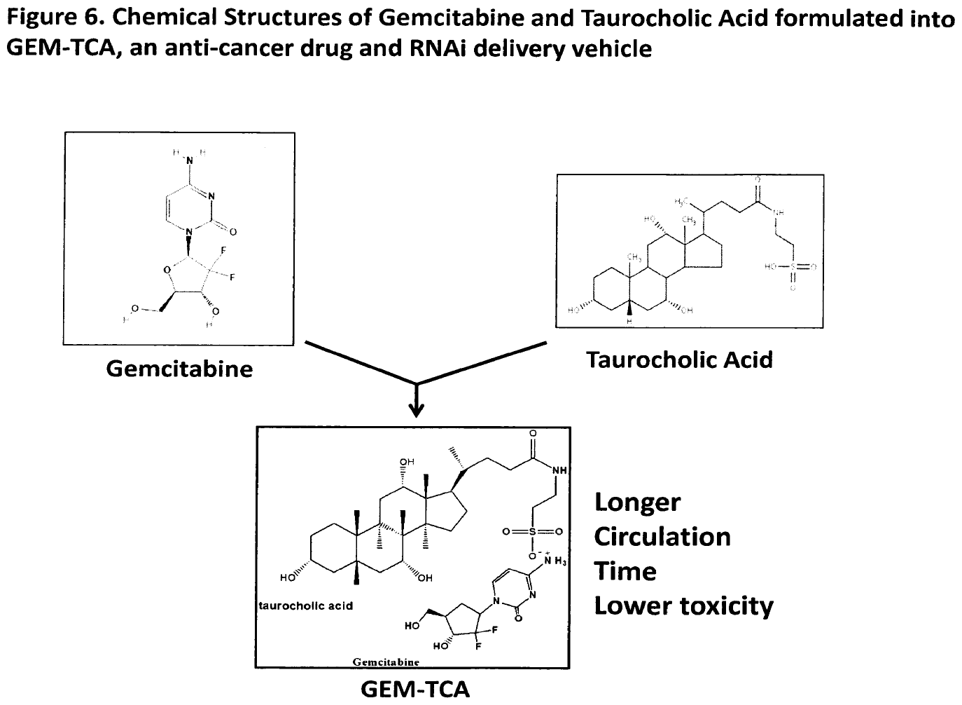

Gemcitabine, (2′-deoxy-2′,2′-difuorocytidine; 1-(4 amino-2-oxo-1H-pyrimidin-1-yl)-2-deoxy-2,2-difluro-D-cytodine; dFdC; CAS No. 95058-81-4; C9HUF2N3O4, Mr 263.2) is an officially monographed substance in the US Pharmacopoeia (Official Monographs, USP 27, 1st Supplement USP NF, page 3060-61, relating to “Gemcitabine Hydrochloride” and “Gemcitabine for Injection”). Gemcitabine has the following chemical structure: Chemical formula: C26H45NO7S; Molar mass: 515.7058 g/mol; Melting point: 125.0° C. (257.0° F.; 398.1 K). The structure of Gembitabine is shown in FIG. 6.

Taurocholic acid is a powerful biological detergent and can be used to dissolve lipids and to free membrane bound proteins. It is a bacteriology culture media ingredient and used in some forms of MacConkey's broth. It can also accelerate lipase activity. It has potential in the manufacture of vaccines and as a vehicle to assist with drug and vaccine delivery. Taurocholic acid is a bile acid and is the product of conjugation of cholic acid with taurine. Its sodium salt is the chief ingredient of the bile of carnivorous animals. It is a deliquescent yellowish crystalline bile acid involved in the emulsification of fats. It occurs as a sodium salt in the bile of mammals. In medical use, it is administered as a cholagogue and choleretic. Hydrolysis of taurocholic acid yields taurine. The structure of Taurocholic acid is shown in the FIG. 6.

The present invention provides compositions of taurocholic acid coordinated with gemcitabine in which the liposome can contain any of a variety of negatively-charged molecules, such as siRNA or miRNA oligos. The complex-forming materials are amphiphilic molecules such as Glycocholic acid, or cholylglycine, or taurolipids, ceramide-1 sulfonates etc. The term “Gemcitabine” as used herein means Gemcitabine free base and Gemcitabine derivatives.

The compositions can be used advantageously in conjunction with secondary therapeutic agents other than gemcitabine, including siRNA and miRNA, antineoplastic, antifungal, antibiotic among other active agents, particularly cisplatin, antisense oligonucleotides, oxaliplatin, paclitaxel, vinorelbine, epirubicin. The invention specifically contemplates methods in which a therapeutically effective amount of the inventive complex in a pharmaceutically acceptable excipient are administered to a mammal, such as a human. We name this newly formulated structure GEM-TCA as shown in FIG. 6.

### Example 6. Formulation of Gemcitabine-Taurocholic Acid (GEM-TCA)

The formulation involves two steps:

Preparation of gemcitabine free base:

Gemcitabine Hydrochloride is the active ingredient in drug products sold under numerous trade names. To prepare the free-base of gemcitabine, add gemcitabine hydrochloride (5.0 g) and potassium carbonate (4.0 g, 1.5 molar equivalents) to a 1.0 L round bottom flask. Then add dichloromethane (350 mL) and ethanol (300 mL). Stir vigorously the contents of the flask at room temperature overnight. Filter the milky white solution with a fritted funnel to a clean bottle. Remove a majority of the solvent by evaporation with the aid of forced dry air. Place the solids under high vacuum for 8 hours at 30° C. Free-base is white solid powder, verification was done by 41-NMR.

Preparation of Gemcitabine-Taurocholic Acid Salt (1:1), Prodrug:

Dissolve 0.30 g (1.139 mmol) of gemcitabine free base in ethyl alcohol (20 mL; 200 proof) at 50° C. In a separate flask, dissolve taurocholic acid (0.58 g; 1.124 mmol) in ethyl alcohol (10 mL; 200 proof). Add TC solution to gemcitabine dropwise. Add 10 mL ethanol and stir solution at 50° C. (˜30 min) until precipitation occurs. Cool the solution at room temperature. Collect precipitated solid by vacuum filtration and allow to dry under vacuum desiccator. As the result, we have the appearance as white solid.

Desirably, the composition and method present one or more of the following advantages: 1) achieve a strong electrostatic interaction between anionic steroid and gemcitabine, 2) avoidance of solubility problems, 3) high stability of gemcitabine—taurocholate complex 4) ability to administer gemcitabine as a bolus or short infusion in a high concentration, 5) prolong half-life of gemcitabine, 6) reduced gemcitabine toxicity, 7) increased therapeutic efficacy of gemcitabine, and 8) modulation of multidrug resistance in cancer cells.

### Example 7. Cytotoxicity Comparison Between GEMZAR® and GEM-TCA

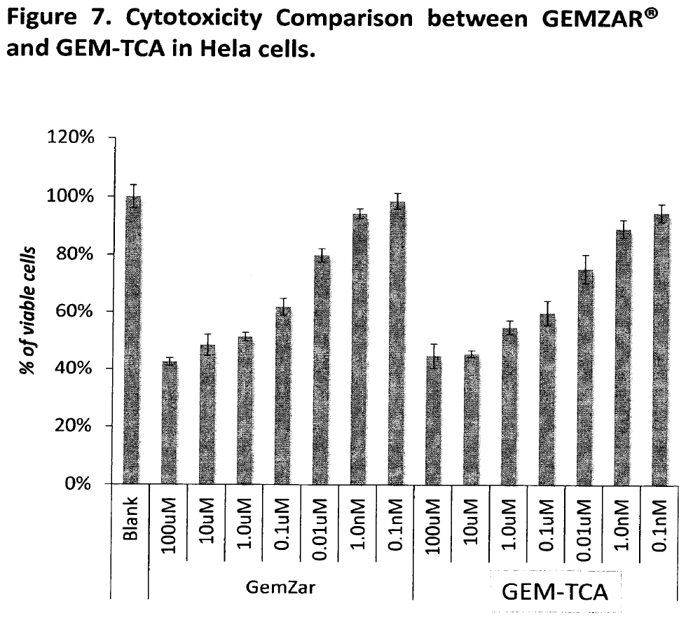

After obtaining the GEM-TCA formulation, we tested its tumor cell killing potency in comparison with GEMZAR®, an approved anticancer drug. 1×10³ HeLa cells were seeded on the wells of 96-well plate on the day before treatment in 150 ul of EMEM supplemented with 10% FBS. On the next day 50 uL of GEMZAR® or GEM-TCA were diluted in the same medium and added to the cells (0.1 nM-100 uM). At 72 h post chemical exposure cytotoxicity was assessed with CellTiter-Glo Luminescent cell viability assay (Promega). Values derived from untreated cells (Blank) were set as 100%. All values represent the mean of ±S.D. of four replicates for each dilution (FIG. 7). Clearly, GEM-TCA has demonstrated the same anti-cancer (tumor cell killing) activity as GEMZAR® does, in a Hela cell culture study with concentrations from 0.1 nM to 100 nM.

### Example 8. Comparison Between GEMZAR® and GEM-TCA in HepG2 and Panc-1 Cell Culture

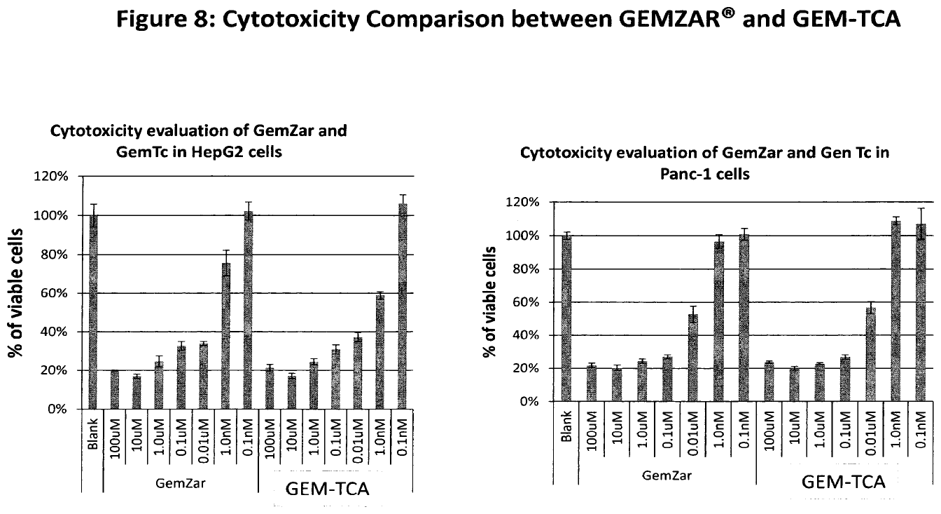

We further compared the tumor cell killing potencies of GEMZAR® and GEM-TCA with HepG2 (a perpetual cell line consisting of human liver carcinoma cells, derived from the liver tissue of a 15-year-old Caucasian male who had a well-differentiated hepatocellular carcinoma) and Panc-1 (a cell line established from a pancreatic carcinoma of ductal origin of a 56-year-old Caucasian male) cell cultures, followed by measurements of cell viability. Cytotoxicity comparison between GEMZAR® and GEM-TCA was conducted with following steps. 2×10³ Panc-1 and HepG2 cells were seeded on the wells of 96-well plate in 150 ul of EMEM/10% FBS. The next day, the medium was supplemented with 0.1 nM-100 uM GEMZAR® or GemTc diluted in the same medium. At 72 h post chemical exposure cytotoxicity was assessed with Cell Titer-Glo Luminescent cell viability assay (Promega). Values derived from untreated cells (Blank) were set as 100%. All values represent the mean of ±S.D. of four replicates for each dilution. Again, GEM-TCA has demonstrated the same tumor cell killing potencies with both HepG2 and Panc-1 cell culture studies at concentrations from 0.1 nM to 100 nM (FIG. 8).

### Example 9. Effect of Forward Transfection with siRNA Specific to mRNA of mTOR Gene on Chemosensitivity of Panc-1 Cells to GEM-TCA

Pancreatic tumor is the most lethal type of digestive cancer with a 5-year survival rate of 5%. Adjuvant chemotherapy remains to be Gemcitabine alone or combined with infusional 5-fluorouracil with radiation therapy. Once pancreatic cancer becomes metastatic, it is uniformly fatal with an overall survival of typically 6 months from diagnosis. Gemcitabine has been the standard in both locally advanced and metastatic disease. The addition of the tyrosine kinase inhibitor erlotinib prolongs median survival for only 2 weeks. While Gemcitabine-based regimens are currently accepted as the standard first-line treatment of patients with locally advanced or metastatic pancreatic adenocarcinoma, there is no consensus regarding treatment in the second-line setting. Recently, two targeted agents, a tyrosine kinase inhibitor Sunitinib and mTOR inhibitor Everolimus have been approved by FDA for pancreatic neuroendocrine tumors.

We have identified potent mTOR specific siRNA through cell culture studies with human breast cancer cell line MDA-MB-231 and mouse CT26 cells, followed by qRT-PCR analyses: mTOR-siRNA:

sense:
5′-r(GGUCUGGUGCCUGGUCUGAUGAUGU)-3′
Antisense:
5′-r(ACAUCAUCAGACCAGGCACCAGACC)-3′

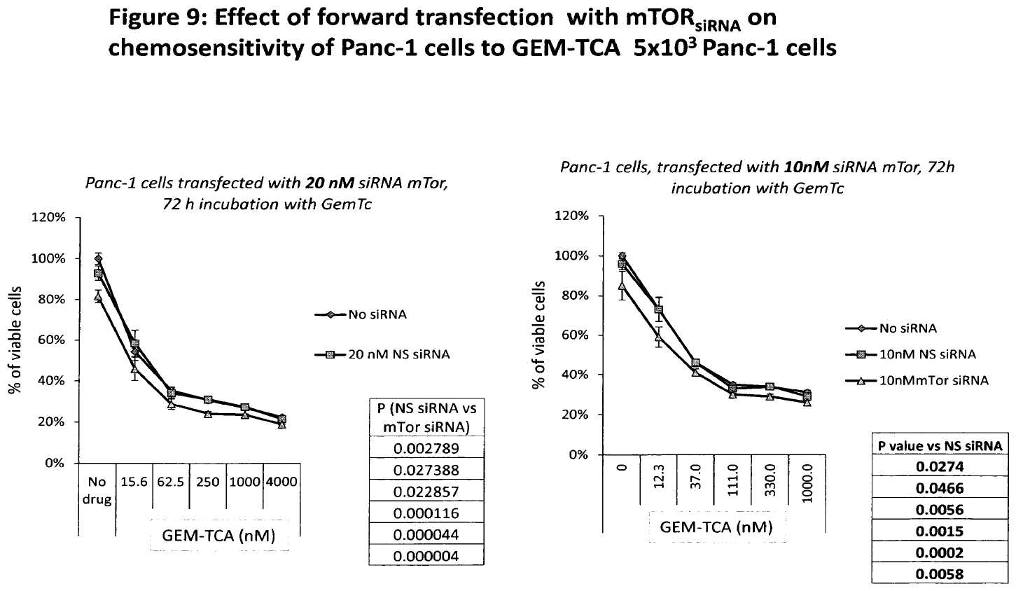

To realize the original hypothesis that the oncogenic gene target knockdown may induce a chemosensitivity of Panc-1 cell toward to GEM-TCA, the experiment was conducted with following procedures. 5×10³ Panc-1 cells were seeded on the wells of 96-well plate in 100 ul of DMEM/10% FBS. The next day cells were transfected with siRNA/Lipofectamine 2000 complexes accordingly to the manufactures' recommendations. In 5-6 h. medium was changed. The next day various concentrations of GEM-TCA are applied to the transfected cells. At 72 h post chemical exposure cytotoxicity was assessed with Cell Titer-Glo Luminescent cell viability assay (Promega). Values derived from untreated cells (Blank) were set as 100%. All values represent the mean of ±S.D. of four replicates for each dilution different from cells transfected with control, not-targeting siRNA (p<0.05, Student's t test). Based on the observation on the FIG. 9, we can see that at two fixed mTOR-siRNA concentrations: 10 nM and 20 nM, the tumor cell killing by GEM-TCA was significantly improved at concentration from 12.3 nM to 1 μM.

### Example 10. Effect of TGF-β1_{siRNA} and mTOR_{siRNA} on the Chemosensitivity of Panc-1 Cells Exposed to Low Dose GEM-TCA

In order to have good understanding of natures of TGF-β1_{siRNA} and mTOR_{siRNA} induced chemosensitivities of Panc-1 cells to GEM-TCA, we have tested these two siRNA duplexes at the fixed concentration of 30 nM, and then cells were further exposed to GEM-TCA at various concentrations from 3.9 nM to 1 μM. The next day, the medium was supplemented with 3.9 nM-1000 nM GemTc diluted in the same medium. At 48 h post chemical exposure cytotoxicity was assessed with Cell Titer-Glo Luminescent cell viability assay (Promega). Values derived from untreated cells (Blank) were set as 100%. All values represent the mean of ±S.D. of four replicates for each dilution. Paired sample two-tailed Student's t-test was used to determine significance. The TGF-β1_{siRNA} was previously identified and validated with multiple in vitro and in vivo assays:

Sense:
5′-r(CCUCAAUUCAGUCUCUCAUCUGCAA)-3′
Antisense:
5′-r(UUGCAGAUGAGAGACUGAAUUGAGG)-3

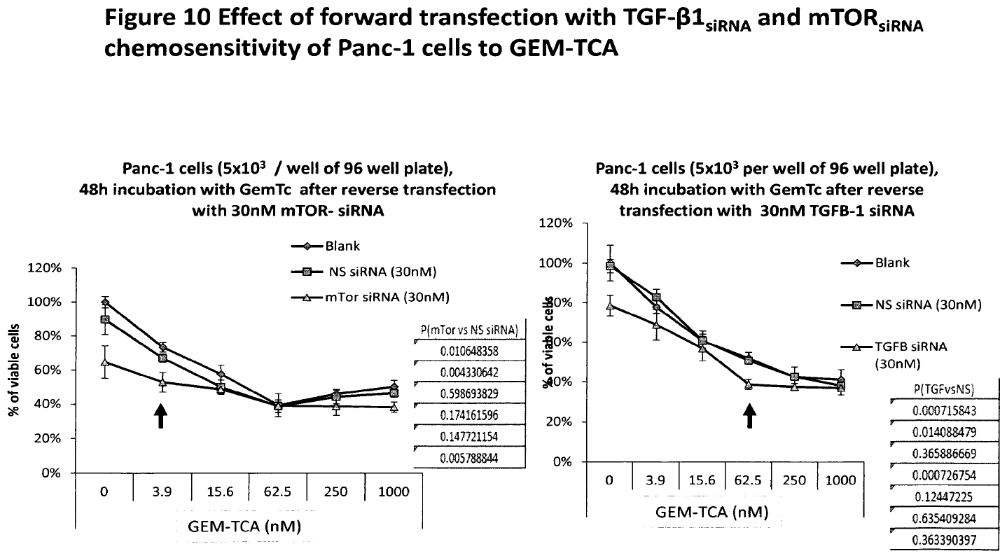

From the observation in FIG. 10, we found both TGF-β1_{siRNA} and mTOR_{siRNA} are able to significantly sensitize Panc-1 cell to GEM-TCA treatment at a low concentration (3.9 nM). When GEM-TCA concentration was increased to 15.6 nM, the sensitization effect was disappeared for mTOR_{siRNA} treatment. However, even when GEM-TCA concentration was increased to 62.5 nM, the sensitization effect was still significant. In both cases, the maximum cell killings were stopped at 60%. Based on these results, we conclude that GEM-TCA has preserved the tumor cell killing property and its anti-tumor activity can be further enhanced by mTOR_{siRNA} or other tumor target silencing siRNAs.

### Example 11. Characterization of GEM-TCA/siRNA Nanoparticles

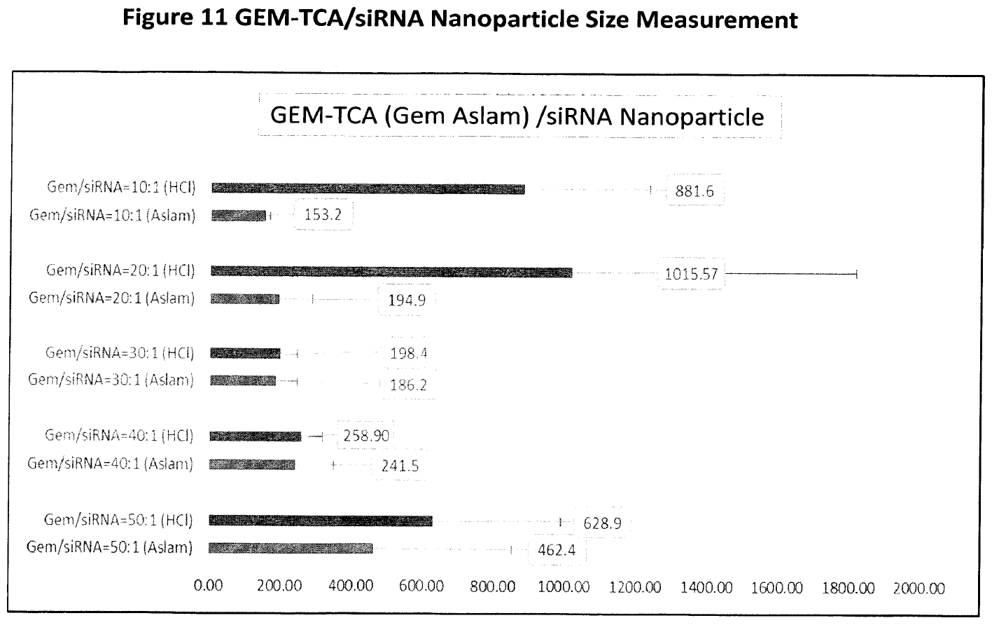

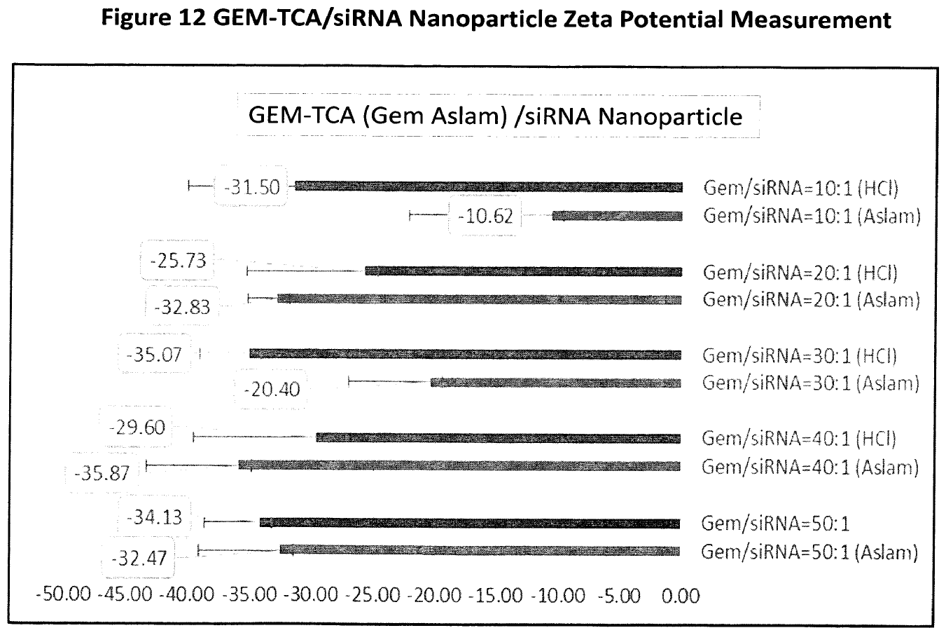

We further measured the particle size and Zeta potential of GEM-TCA/siRNA formulation at a ratio of 10/1, or 20/1, or 30/1, or 40/1, or 50/1. As the results, when GEM-TCA/siRNA at 10/1 ratio, the particle sizes in average is about 153.2 nm (FIG. 11) with Zeta potential about −10.62 (FIG. 12). Therefore, GEM-TCA is able to package siRNA into nanoparticles with a ratio of molecular weight 10/1.

### Example 12. Design of a Conjugation Strategy for Gemcitabine and HKP

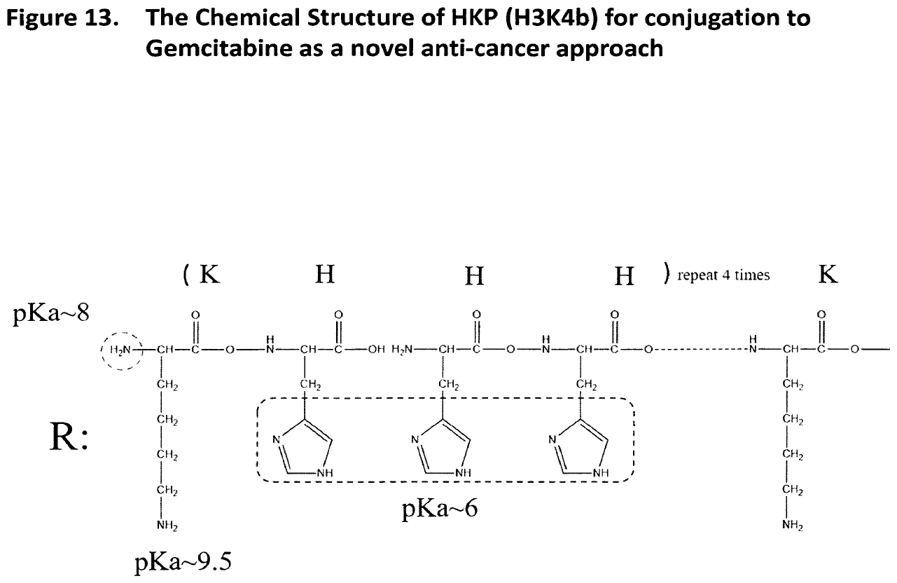

As a polyamine, residue repeating and branched peptide, HKP is very hard to modified. There are three kinds of functional amine (excluding the amine in peptide bond): 48 imidazole groups, 20 epsilon-amine, and 5 N-terminal alpha-amine. If one wants to modify HKP through those amine, they will interfere each other, and one will finally produce multiple intermediates with variable branches. We found that there is one special amine at the C-terminal end of the HKP, which is different from all the other functional amine groups. It was the position used to be hydroxyl (—OH) in C-terminal carboxyl, but replaced by amine in HKP, called amide (FIG. 13). It has a special character that the lone pair of electrons at nitrogen relocated into carbonyl, finally form C═N double bond, and a hydroxyl. Actually, the amide will be acid catalyzed hydrolysis into carboxyl. That means the only “C-terminal” of HKP turn back carboxyl group at acid conditions. It becomes the unique breakout that we can take advantage of for modification. We can modify HKP through the C-terminal carboxyl group.

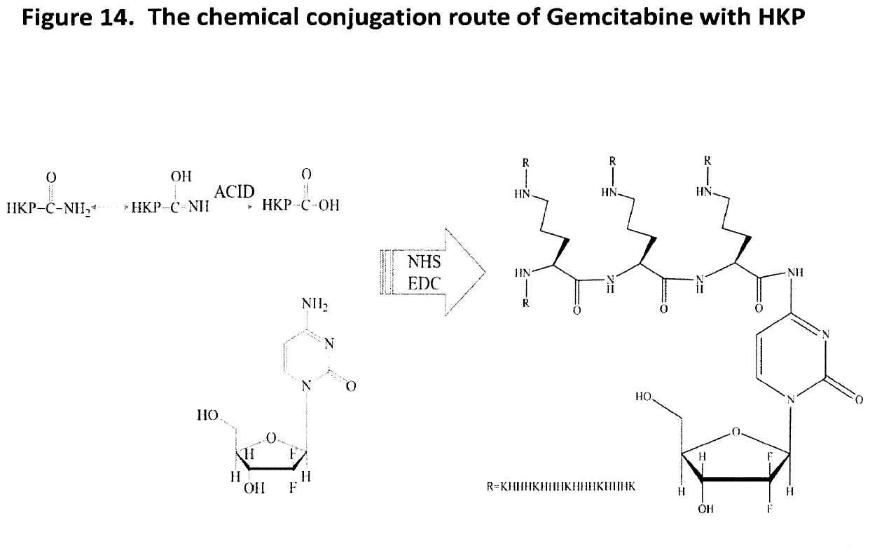

Gemcitabine is a nucleoside analogue. Most chemical modifications of gemcitabine are exclusively through two sites, 4-(N) and 5′-(OH), and there are various gemcitabine derivatives developed. As a prodrug, modification through those two sites allowed gemcitabine to be released as active drug within the body, and improve the delivery efficiency. As proposed in FIG. 14, we decided to select EDC-NHS chemistry as the strategy to conjugate HKP and Gemcitabine. This is carbodiimide crosslinker chemistry. EDC (also called EDAC) is 1-ethyl-3-(−3-dimethylaminopropyl) and NHS is N-hydroxysuccinimide.

The advantage of using EDC-NHS chemistry:

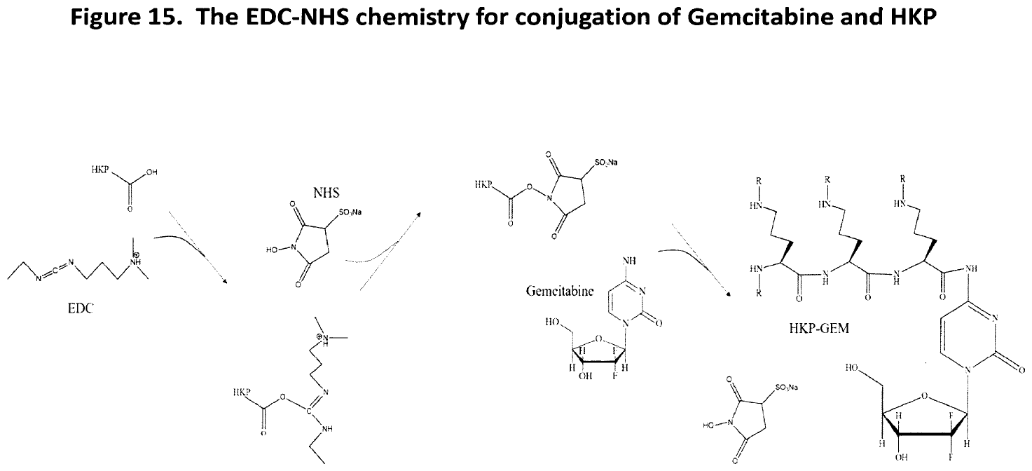

1. EDC-NHS reaction occurs most effectively at acid condition.
2. HKP will generate carboxyl group under acid condition.
3. EDC-NHS reaction prefer —NH₂ rather than —NH₃⁺.
4. —NH₂ of Gemcitabine outstands from interfering amines of HKP at acid conditions due to the low pKa value (˜2.8), which make Gemcitabine conjugate with HKP instead of HKP self-conjugation (FIG. 15).

### Example 13. Characterization of GEM-HKP Structure and Molecular Weight

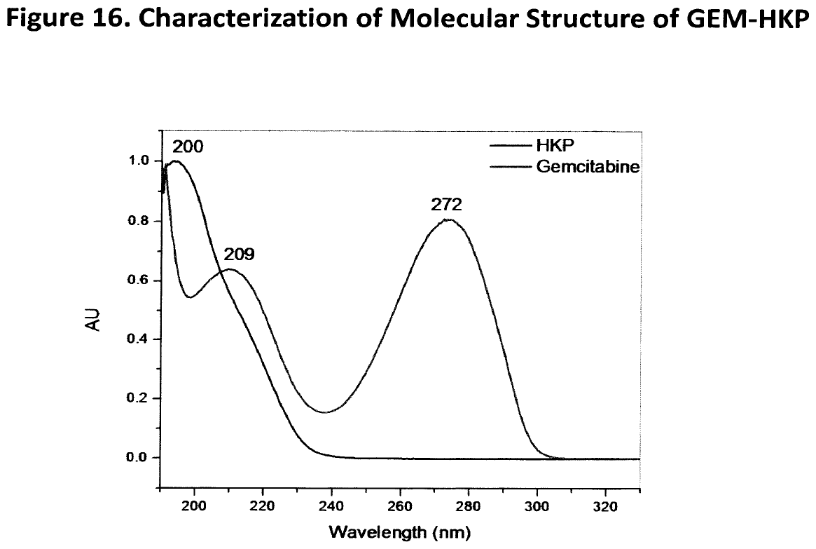

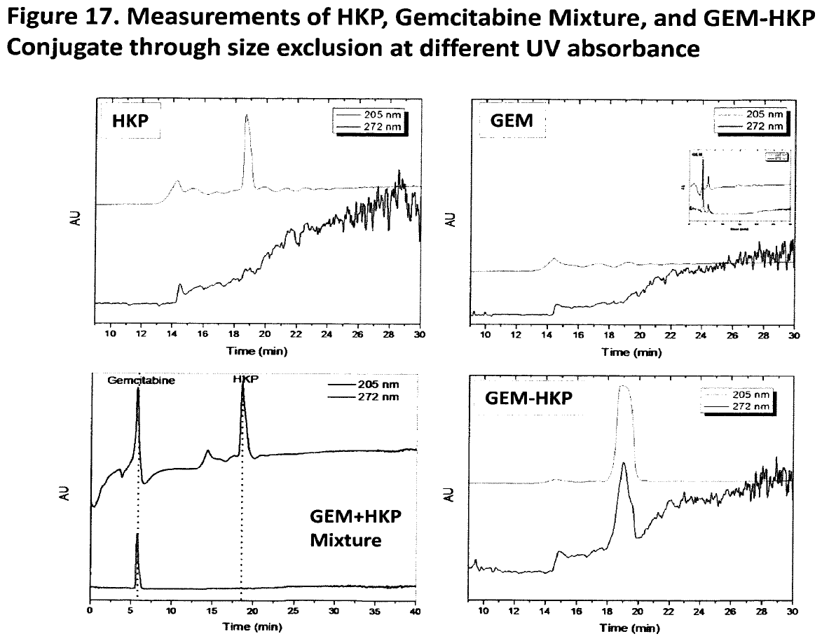

As seen in FIG. 16, the HKP molecule has a characteristic UV absorbance peak at around 200 nm, which attributes mainly to histidine, whereas Gemcitabine has two peaks, representing sugar at 209 nm and 272 nm for cytosine respectively. So we selected peak wavelength of 272 nm as indication of Gemcitabine, and 205 nm as indication of HKP. Then we ran the HPLC assay for pure HKP and Gemcitabine as shown in FIG. 17. Due to the huge difference of molecule weights between HKP (9.6 kD) and Gemcitabine (236D), they came out from the column at different time points. HKP peak appeared at ˜19 min, whereas Gemcitabine peak at ˜5 min. Gemcitabine has no absorbance at ˜19 min at all. However, when GEM-HKP was measured, this single compound exhibits the absorbance at both 205 nm and 272 nm, and shows two picks at ˜19 min together.

After conjugating HKP and Gemcitabine, the as-produced compound showed two strong peaks at both wavelengths of 272 nm and 205 nm, at the same time point of ˜19 min. Comparing with HKP, the Gemcitabine is much smaller molecule (40× smaller), as shown in the proposed reaction mechanism, one molecule Gemcitabine added on HKP will not retard the HKP peak position much. Also, although Gemcitabine has absorbance at ˜205 nm as well, if under equal-molar level, its absorbance is negligible comparing to HKP. Furthermore, we didn't find any other strong peaks at longer or shorter time point (from 0 to 60 min).

Based on the HPLC and UV results, we can make the conclusions below:

1. The proposed HKP-Gemcitabine (HKP-GEM) compound is synthesized successfully.
2. The new compound has one gemcitabine binding with one HKP.
3. No significant side product was observed.

### Example 14. GEM-HKP/siRNA Nanoparticle Formulation Property

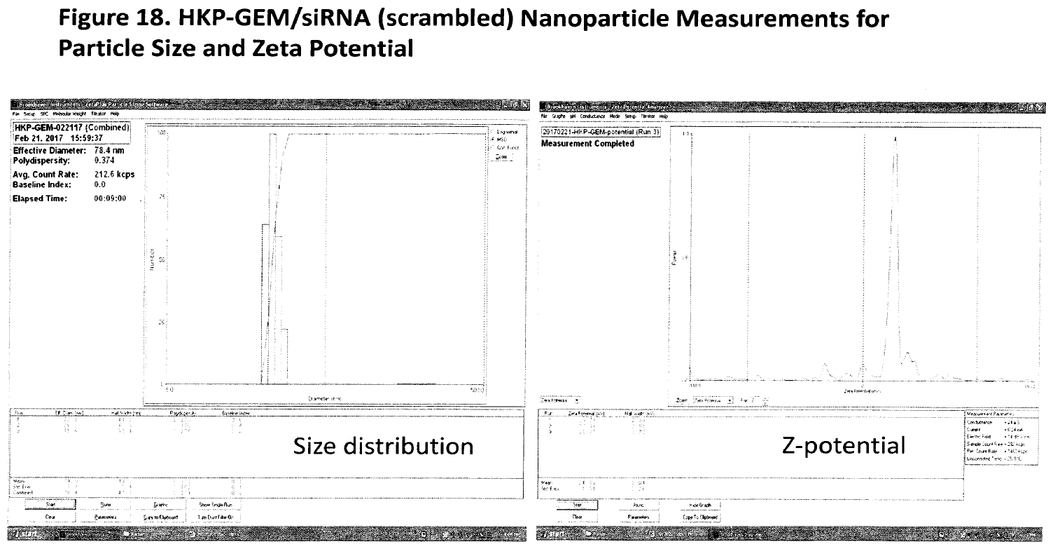

We further measure the physiochemical properties (particle sizes and Zeta potential) of nanoparticle formation when HKP-GEM aqueous solution and siRNA aqueous solution mixed together at a 4:1 ratio. The scrambled siRNA was used with GEM-HKP to form nanoparticles and the original HKP was used as positive control under the same condition. The size and Zeta potential of the nanoparticles were measured using Brookhaven 90Plus Nanosizer. As indicated in the FIG. 16, the average particles sizes of GEM-HKP is 79 nm with Zeta potentials of 25 mV. The nanoparticle of GEM-HKP/siRNA has similar Zeta potential with that of HKP/siRNA, but different particle sizes (FIG. 18). However, since this is new compound, the optimum ratio may change a little bit, which may be worked out later. Based on the nanosizer results, the nanoparticle formation ability of the new compound HKP-GEM was verified.

TABLE A
Nanoparticle characterization

| | Size (nm) | z-potential (mV) |
|---|---|---|
| HKP | 125 | 26 |
| HKP-GEM | 79 | 25 |

### Example 15. GEM-HKP Delivers siRNA into Panc-1 Cells

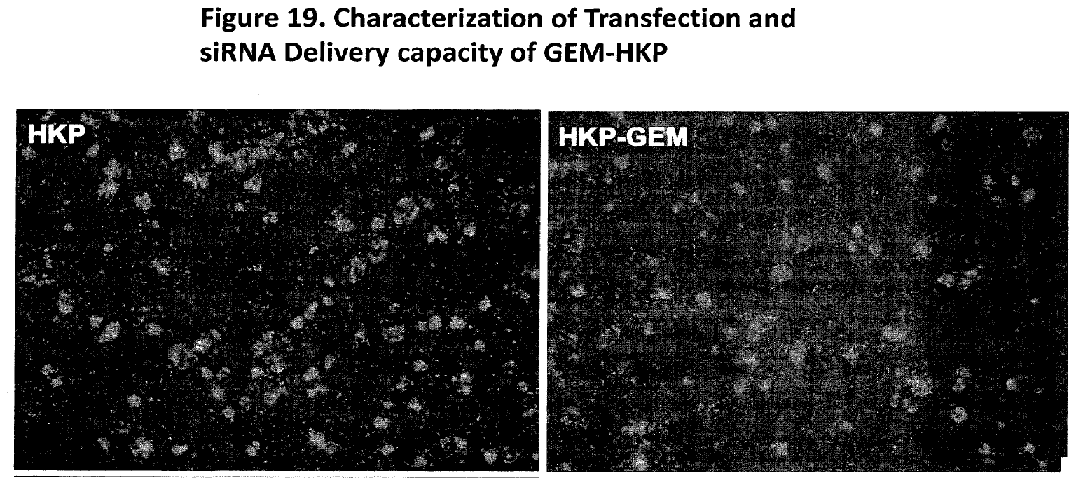

We then used AF488 siRNA (scrambled siRNA modified with Fluorescent AF488) as a reporter to form nanoparticles together with GEM-HKP to evaluate their capability for in vitro siRNA transfection. HKP-siRNA nanoparticle was used as control. As shown in FIG. 19, our new compound, GEM-HKP, has an ability to deliver siRNA into the cells with the similar efficiency with HKP. Panc-1 cell line was used as the model for this evaluation.

### Example 16. GEM-HKP Exhibits Tumor Cell Killing Activity

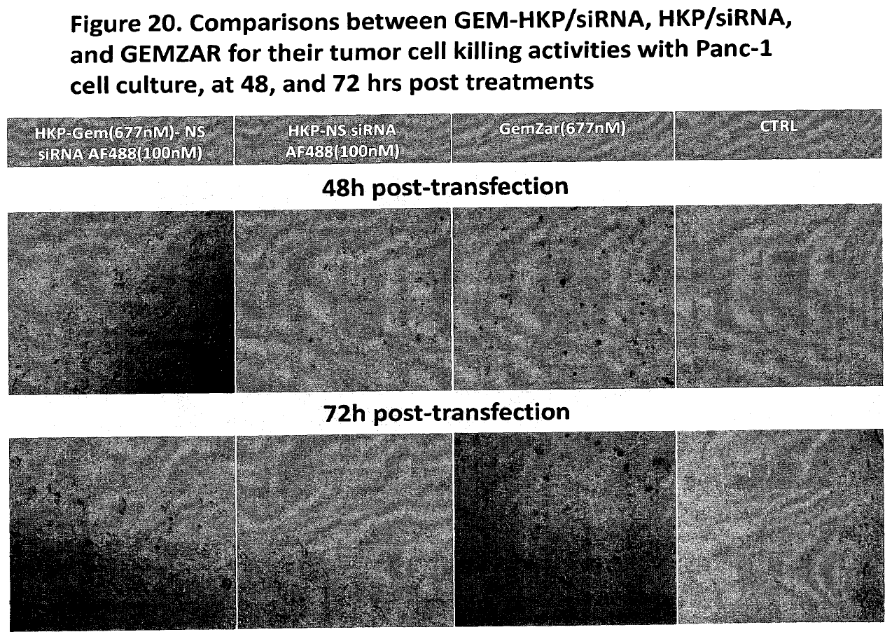

Based on the observations in FIG. 19, we moved further to test the GEM-HKP for its cytotoxic activity for killing tumor cells. Non-coding AF488 labeled siRNA was transfected into Panc-1 cells with HKP or GEM-HKP at a ratio of carrier:siRNA as 4.5:1. Twenty-four hours post-transfection, medium containing siRNA and transfection agent or drug alone were replaced with fresh medium. At 48 hours and 72 hours post transfection, the images of cell growth were taken for evaluation of cell killing (FIG. 20). Although the cell killing activity was not very clear at 24 hours post transfection, the GEM-HKP carried siRNA nanoparticle has demonstrated potent cell killing activity. The result suggests that GEM-HKP is able to preserve the properties of siRNA delivery (HKP function) and tumor cell killing (Gemcitabine function). Therefore, GEM-HKP represents a novel anti-tumor agent while is able to delivery therapeutic siRNA drugs.

### Example 17. GEM-TAC is Active Tumor Growth Inhibitor in A549 Xenograft Tumor Model More Potent than GemZar

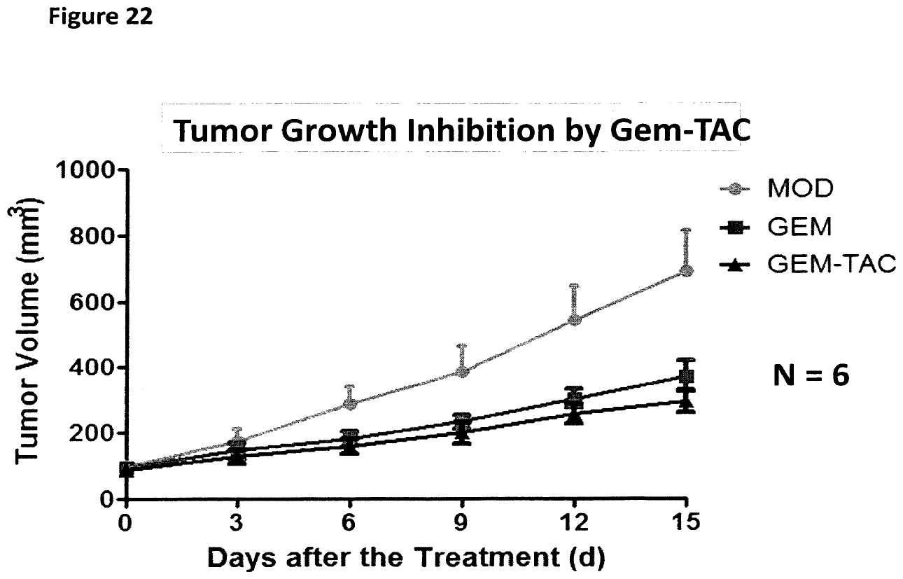

The Tumor Inhibition Test with A549 (Lung Cancer) Cell Xenograft Mouse Model has demonstrated that MOD is the tumor model group without treatment. GEM is the tumor model group treated with GemZar. GEM-TCA is the tumor model group treated with Gemcitabine-Taulichoric Acid formulation. Cohort group N=6. GemZar and GEM-TAC were used with the same dosage (FIG. 22).

### Example 18. GEM-TAC is Active Tumor Growth Inhibitor in PANC-1 Xenograft Tumor Model More Potent than GemZar

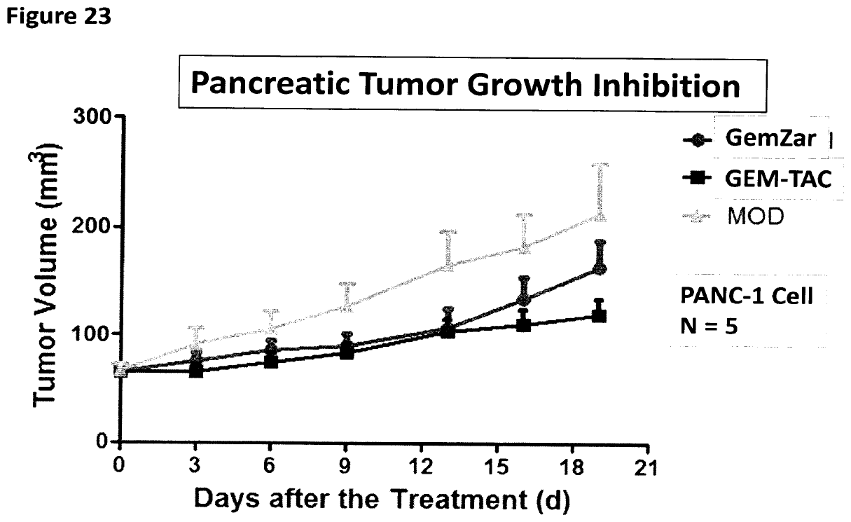

The Tumor Inhibition Test with PANC-1 (Pancreatic Cancer) Cell Xenograft Mouse Model has demonstrated that MOD is the tumor model group without treatment. GEM is the tumor model group treated with GemZar. GEM-TCA is the tumor model group treated with Gemcitabine-Taulichoric Acid formulation. Cohort group N=6. GemZar and GEM-TAC were used with the same dosage (FIG. 23, 24, 25).

### Example 19. GEM-TAC is Able to Enhance Antitumor Activity in Combination with STP302 in Lovo Cell Xenograft Tumor Model

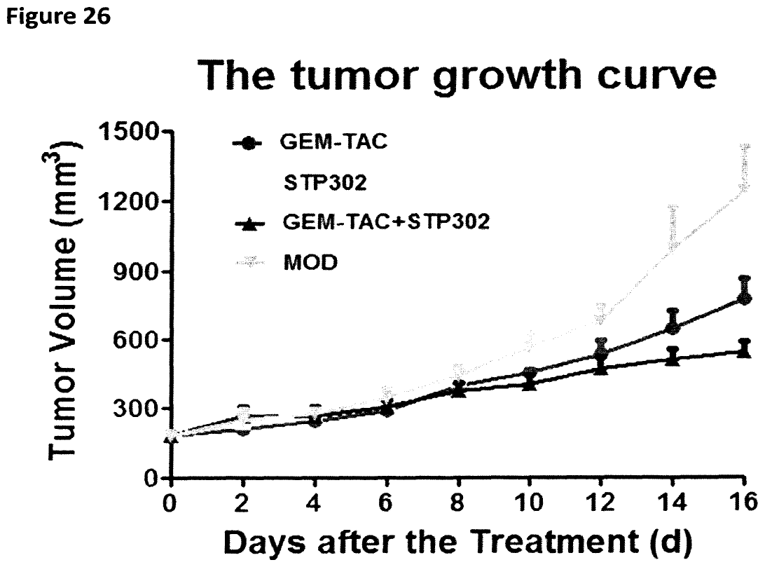

The Tumor Inhibition Test with Lovo cell (Colon Cancer) Cell Xenograft Mouse Model has demonstrated that MOD is the tumor model group without treatment. STP302 is a miRNA therapeutic candidate with mir150/HKP formulation. GEM-TAC is the tumor model group treated with Gemcitabine-Taulichoric Acid formulation. Cohort group N=6GEM-TAC+STP302 combination resulted in better efficacy than their individual use (FIG. 26, 27).

### Example 20. GEM-TAC is Able to Enhance Antitumor Activity in Combination with STP302 in Lovo Cell Xenograft Tumor Model

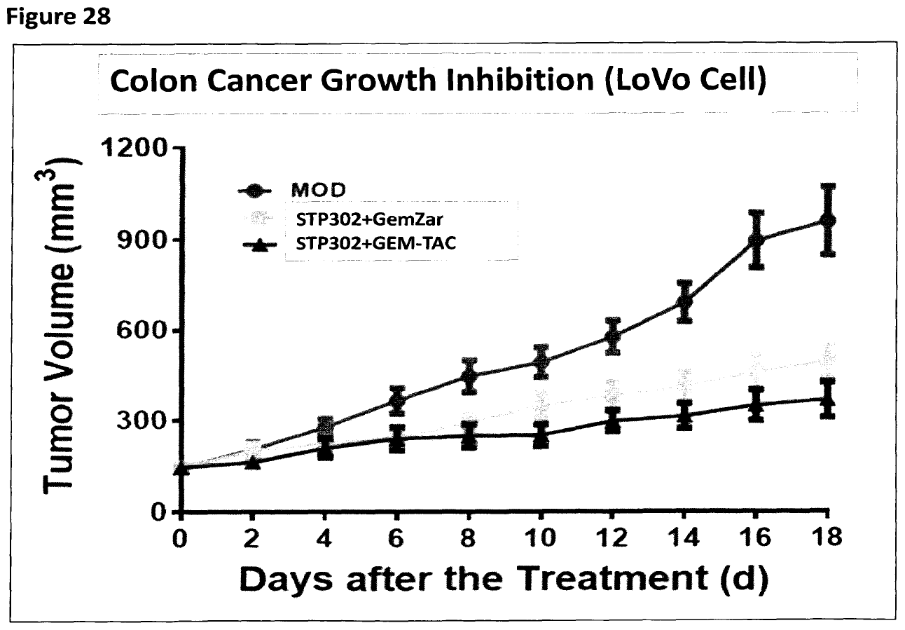

The Tumor Inhibition Test with Lovo cell (Colon Cancer) Cell Xenograft Mouse Model has demonstrated that MOD is the tumor model group without treatment. MOD is the tumor model group without treatment. GEM is the tumor model group treated with GemZar. GEM-TAC is the tumor model group treated with Gemcitabine-Taulichoric Acid formulation. Cohort group N=8. GemZar and GEM-TAC were used with the same dosage. There is significant difference between the therapeutic benefits of GemZar and GEM-TAC (FIG. 28, 29).

### Example 21. Potent siRNA Sequences were Selected Against Human PDL-1 Gene Using Caski Cell Culture Study

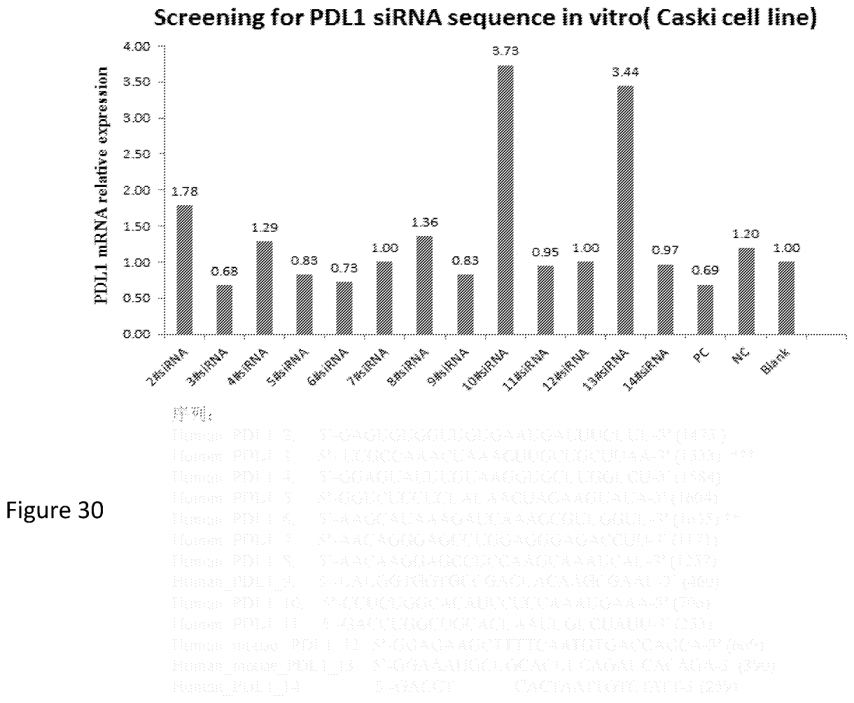

Multiple siRNA sequences were screened for inhibition of PDL-1 gene expression using human cervical cancer cell line, Caski cell culture. Positive siRNA sequences were marked with stars (FIG. 30, 31). Human_PDL1_3. 5′-UCGCCAAACUAAACUUGCUGCUUAA-3′ (1533); Human_PDL1_6. 5′-AAGCAUAAAGAUCAAACCGUUGGUU-3′ (1635) **.

### Example 22. Potent siRNA Sequences were Selected Against Human PDL-2 Gene Using Caski Cell Culture Study

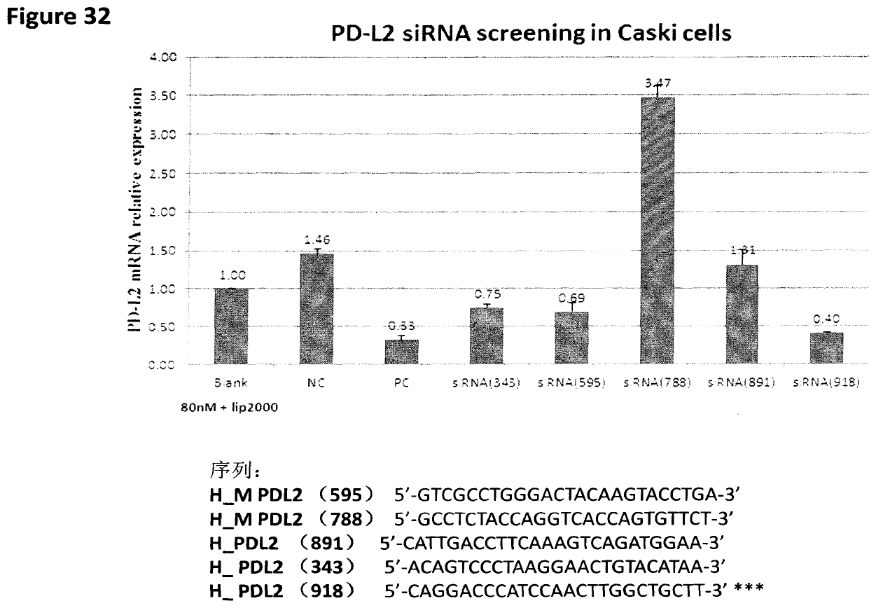

Multiple siRNA sequences were screened for inhibition of PDL-2 gene expression using human cervical cancer cell line, Caski cell culture. Positive siRNA sequences were marked with stars (FIG. 32, 33). Human_PDL1_6. 5′-AAGCAUAAAGAUCAAACCGUUGGUU-3′ (1635) ***; H_PDL2 (918) 5′-CAGGACCCATCCAACTTGGCTGCTT-3′ ***.

TABLE 1
Sense Sequences of siRNA Inhibitors

| | |
|---|---|
| EGFR: | 5′-GAUCAUGGUCAAGUGCUGGAUGAUA-3′ |
| VEGF: | 5′-CUGUAGACACACCCACCCACAUACA-3′ |
| PDGF: | 5′-GCCUGCUGCUCCUCGGCUGCGGAUA-3′ |
| RAF1: | 5′-GCCUGCUGCUCCUCGGCUGCGGAUA-3′, |
| VER2: | 5′-CAUGGAAGAGGAUUCUGGACUCUCU-3′ |

TABLE 2
Sense Sequences of siRNA Oligos:

| | |
|---|---|
| EGFR: | 5′-GAUCAUGGUCAAGUGCUGGAUGAUA-3′ |
| VEGF: | 5′-CUGUAGACACACCCACCCACAUACA-3′ |
| PDGF: | 5′-GCCUGCUGCUCCUCGGCUGCGGAUA-3′ |
| RAF1: | 5′-GCCUGCUGCUCCUCGGCUGCGGAUA-3′ |
| VER2: | 5′-CAUGGAAGAGGAUUCUGGACUCUCU-3′ |

### REFERENCES

1. Makrilia, N., K. N. Syrigos, M. W. Saif. Updates on Treatment of Gemcitabine-Refractory Pancreatic Adenocarcinoma. Journal of Pancreas, 2011, 12(4):351-354.
2. Saif M. M. Pancreatic Neoplasm in 2011, An Update. Journal of the Pancreas, 2011, 12(4): 316-321.
3. Park J K, Henry J C, Jiang J, Esau C, Gusev Y, Lerner M R, Postier R G, Brackett D J, Schmittgen T D. miR-132 and miR-212 are increased in pancreatic cancer and target the retinoblastoma tumor suppressor. Biochem Biophys Res Commun. 2010, 406(4):518-23.
4. Greither T, Grochola L F, Udelnow A, Lautenschlager C, Wiirl P, Taubert H. Elevated expression of microRNAs 155, 203, 210 and 222 in pancreatic tumors is associated with poorer survival. Int J Cancer. 2010 Jan. 1; 126(1):73-80.
5. Habbe N, Koorstra J B, Mendell J T, Offerhaus G J, Ryu J K, Feldmann G, Mullendore, M E, Goggins M G, Hong S M, Maitra A. MicroRNA miR-155 is a biomarker of early pancreatic neoplasia. Cancer Biol Ther. 2009, 8(4):340-6.
6. Gironella M, et al. Tumor protein 53-induced nuclear protein 1 expression is repressed by miR-155, and its restoration inhibits pancreatic tumor development. Proc Natl Acad Sci USA. 2007, 104(41):16170-5.
7. Han Z B, Chen H Y, Fan J W, Wu J Y, Tang H M, Peng Z H. Up-regulation of microRNA-155 promotes cancer cell invasion and predicts poor survival of hepatocellular carcinoma following liver transplantation. J Cancer Res Clin Oncol. 2012, 138(1):153-61.
8. Donnem T, Eklo K, Berg T, Sorbye S W, Lonvik K, Al-Saad S, Al-Shibli K, Andersen S, Stenvold H, Bremnes R M, Busund L T. Prognostic impact of MiR-155 in non-small cell lung cancer evaluated by in situ hybridization. J Transl Med. 2011, 9:6.
9. Joseph J. LaConti., Anton Wellstein, et al. Tissue and Serum microRNAs in the KrasG12D Transgenic Animal Model and in Patients with Pancreatic Cancer. Plos ONE, 2011, Vol. 6(6): 1-9.
10. Bhela S, Lu P Y, Rouse B T. et al. Role of miR-155 in the pathogenesis of herpetic stromal keratitis. Am J Pathol. 2015 April; 185(4):1073-84.
11. Ying-Ying Lu, Da-Dao Jing, Ming Xu, Kai Wu, Xing-Peng Wang. Anti-tumor activity of erlotinib in the BxPC-3 pancreatic cancer cell line. World J Gastroenterol. 2008. 14(35): 5403-5411.
12. Guopei Luo, et al. RNA interference of MBD1 in BxPC-3 human pancreatic cancer cells delivered by PLGA-poloxamer nanoparticles. Cancer Biology & Therapy. 2009. 8(7):1-5.
13. Kyonsu Son, Shuichi Fujioka, Tomonori Lida, Kenei Furukawa, Tetsuji Fujita, Hisashi Yamada, Paul J. Chiao And Katsuhiko Yanaga. Doxycycline Induces Apoptosis in PANC-1 Pancreatic Cancer Cells. Anticancer Research, 2009. 29: 3995-4004.
14. Weibo Niu, Xiangqun Liu, Zhaoyang Zhang, Kesen Xu, Rong Chen, Enyu Liu, Jiayong Wang, Cheng Peng And Jun Niu. Effects of αvβ6 Gene Silencing by RNA Interference in PANC-1 Pancreatic Carcinoma Cells. Anticancer Research. 2010. 30:135-442.
15. Chuan Ding, Muhammad Khan, Bin Zheng, Jingbo Yang, Lili Zhong and Tonghui Mal. Casticin induces apoptosis and mitotic arrest in pancreatic carcinoma PANC-1 cells. African Journal of Pharmacy and Pharmacology. 2012. 6(6):412-418.
16. Leng, Q., P Scaria, P Y Lu, Woodle M C, and A. J. Mixson: Systemic delivery of HK Raf-1siRNA Polyplexes Inhibits MDA-MB-435 Xenografts. Cancer Gene Therapy. 2008, 1-11.
17. Yan, Z W, P Y Lu and L Y Li et al. The Human Rhomboid Family-1 Gene RHBDF1 Is Essential to Cancer Cells Survival and Critical in EGFR Activation. Molecular Cancer Therapeutics, 2008; 7(6):1355-64.
18. Raymond M. Schiffelers, P Y Lu, Puthupparampil V. Scaria, and Martin C. Woodle, et al. (2004): Cancer siRNA Therapy by Tumor Selective Delivery With Ligand-Targeted Sterically-Stabilized Nanoparticle. Nucleic Acid Research, Vol. 32, No. 19.
19. Hertel, L. W., Kroin, J. S., Misner, J. W. & Tustin, J. M. Synthesis of 2-deoxy-2,2-difluoro-D-ribose and 2-deoxy-2,2′-difluoro-D-ribofuranosyl nucleosides. J. Org. Chem. 53, 2406-2409 (1988).
20. Guo Z w, Z. & Gallo, J. M. Selective Protection of 2′,2′-Difluorodeoxycytidine (Gemcitabine). J. Org. Chem. 64, 8319-8322 (1999).
21. Moysan, E., Bastiat, G. & Benoit, J.-P. Gemcitabine versus Modified Gemcitabine: A Review of Several Promising Chemical Modifications. Mol. Pharm. 10, 430-444 (2013)

The disclosures of all publications identified herein, including issued patents and published patent applications, and all database entries identified herein by url addresses, accession numbers, or otherwise, are incorporated herein by reference in their entirety.

Although this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

What is claimed is:

1. A pharmaceutical composition comprising a gemcitabine (GEM) derivative and a RNA interference (RNAi) trigger.

2. The composition of claim 1, wherein the gemcitabine derivative comprises a gemcitabine molecule in electrostatic attraction with a taurocholic acid (TCA) molecule.

3. The composition of claim 1, wherein the gemcitabine derivative comprises a chemical conjugate comprising a gemcitabine molecule and a Histidine-Lysine Polymer (HKP).

4. The composition of any one of claims 1-3, wherein the RNAi trigger comprises a small interfering RNA (siRNA) oligo, a micro RNA (miRNA) oligo, or an antagomir oligo, for activating a RNAi effect in a mammalian cell.

5. The composition of claim 4, wherein the mammalian cell is a human cell.

6. The composition of claim 4 or claim 5, wherein the siRNA oligo has specific sequence homology to mTOR gene mRNA and has an inhibitory activity to mTOR gene expression.

7. The composition of claim 4 or claim 5, wherein the siRNA oligo has specific sequence homology to mTOR gene mRNA: mTOR-siRNA: sense, 5′-r(CACUACAAAGAACUGGAGUUCCAGA)-3′, antisense, 5′-r(UCUGGAACUCCAGUUCUUUGUAGUG)-3′, and has an inhibitory activity to mTOR gene expression.

8. The composition of claim 4 or claim 5, wherein the siRNA oligo has specific sequence homology to TGF-β1 gene mRNA and has an inhibitory activity to TGF-β1 gene expression.

9. The composition of claim 4 or claim 5, wherein the siRNA oligo has specific sequence homology to TGF-β1 gene mRNA, TGF-β1-siRNA: sense, 5′-r(CCCAAGGGCUACCAUGCCAACUUCU)-3′, antisense, 5′-r(AGAAGUUGGCAUGGUAGCCCUUGGG)-3′, and has an inhibitory activity to TGF-β1 gene expression.

10. The composition of claim 4 or claim 5, wherein the siRNA oligo has specific sequence homology to COX-2 gene mRNA and has an inhibitory activity to COX-2 gene expression.

11. The composition of claim 4 or claim 5, wherein the siRNA oligo has specific sequence homology to COX-2 gene mRNA, COX-2-siRNA: sense, 5′-r(GGUCUGGUGCCUGGUCUGAUGAUGU)-3′, anti sense, 5′-r(ACAUCAUCAGACCAGGCACCAGACC)-3′, and has an inhibitory activity to COX-2 gene expression.

12. The composition of claim 1 further comprising a second RNAi trigger different from the first.

13. The composition of claim 4 or claim 5, wherein the miRNA oligo comprises or has homology to miR-132, miR-150, or miR-155.

14. The composition of claim 4 or claim 5, wherein the antagomir comprises or has homology to antagomir-132, antagomir-150, or antagomir-155.

15. The composition of claim 2, wherein the taurocholic acid comprises a deoxycholic acid with taurine.

16. The composition of claim 2 or claim 3, wherein the gemcitabine comprises gemcitabine free base.

17. The composition of claim 2, wherein the GEM and TCA are in a mole ratio about 0.0:0.1 to 1.0:2.0.

18. The composition of claim 3, wherein the GEM and HKP are chemically conjugated into GEM-HKP with EDC-NHS chemistry.

19. The composition of any one of claim 1, 2, 4, or 5, wherein the GEM-TCA can be administered as a chemo-drug for cancer treatment on its own or can package RNAi or DNA oligos as a combination therapeutic for cancer treatment.

20. The composition of any one of claim 1, 3, 4, or 5, wherein the GEM-HKP can be administered as a chemo-drug for cancer treatment on its own or can package RNA or DNA oligos as a combination therapeutic for cancer treatment.

21. The composition of any one of claim 4, 5, 19, or 20, wherein the siRNA oligo comprises a sequence from Table 1.

22. The composition of any one of claim 4, 5, 19, or 20, wherein the siRNA oligo comprises a sequence from Table 2.

23. The composition of any one of the preceding claims further comprising a pharmaceutically acceptable carrier.

24. A pharmaceutical composition comprising a gemcitabine molecule and a taurocholic acid molecule.

25. The composition of claim 24, wherein the taurocholic acid comprises a deoxycholic acid with taurine.

26. The composition of claim 24 or claim 25, wherein the gemcitabine comprises gemcitabine free base.

27. A pharmaceutical composition comprising a gemcitabine molecule and a Histidine-Lysine Polymer.

28. The composition of claim 27, wherein the gemcitabine comprises gemcitabine free base.

29. The composition of any one of claims 24-28 further comprising a RNA interference trigger.

30. The composition of claim 29 further comprising a second RNAi trigger different from the first.

31. The composition of claim 29 or 30, wherein the RNA interference trigger is selected from the group consisting of a small interfering RNA (siRNA) oligo, a micro RNA (miRNA) oligo, or an antagomir oligo.

32. The composition of claim 31, wherein the siRNA oligo has specific sequence homology to mTOR gene mRNA and has an inhibitory activity to mTOR gene expression.

33. The composition of claim 31, wherein the siRNA oligo has specific sequence homology to mTOR gene mRNA: mTOR-siRNA: sense, 5′-r(CACUACAAAGAACUGGAGUUCCAGA)-3′, antisense, 5′-r(UCUGGAACUCCAGUUCUUUGUAGUG)-3′, and has an inhibitory activity to mTOR gene expression.

34. The composition of claim 31, wherein the siRNA oligo has specific sequence homology to TGF-β1 gene mRNA and has an inhibitory activity to TGF-β1 gene expression.

35. The composition of claim 31, wherein the siRNA oligo has specific sequence homology to TGF-β1 gene mRNA, TGF-β1-siRNA: sense, 5′-r(CCCAAGGGCUACCAUGCCAACUUCU)-3′, antisense, 5′-r(AGAAGUUGGCAUGGUAGCCCUUGGG)-3′, and has an inhibitory activity to TGF-β1 gene expression.

36. The composition of claim 31, wherein the siRNA oligo has specific sequence homology to COX-2 gene mRNA and has an inhibitory activity to COX-2 gene expression.

37. The composition of claim 31, wherein the siRNA oligo has specific sequence homology to COX-2 gene mRNA, COX-2-siRNA: sense, 5′-r(GGUCUGGUGCCUGGUCUGAUGAUGU)-3′, anti sense, 5′-r(ACAUCAUCAGACCAGGCACCAGACC)-3′, and has an inhibitory activity to COX-2 gene expression.

38. The composition of claim 31, wherein the miRNA oligo comprises or has homology to miR-132, miR-150, or miR-155.

39. The composition of claim 31, wherein the antagomir comprises or has homology to antagomir-132, antagomir-150, or antagomir-155.

40. The composition of any one of claims 24-39 further comprising a pharmaceutically acceptable carrier.

41. A method of treating cancer in a mammal or inhibiting the growth of neoplastic or tumor cells in a mammal comprising the step of administering a therapeutically effective amount of the composition of any one of claims 1-40 to the mammal.

42. A method of inducing apoptosis of neoplastic or tumor cells in a mammal comprising the step of administering an effective amount of the composition of any one of claims 1-40 to the mammal.

43. A method of enhancing chemosensitivity of a mammal with cancer to GEM comprising the step of administering an effective amount of the composition of any one of claims 1-40 to the mammal.

44. The method of any one of claims 41-43, wherein the cancer is pancreatic cancer.

45. The method of claims 41-44, wherein the mammal is a laboratory animal.

46. The method of claims 41-44, wherein the mammal is a human.

47. The composition of claim 24, wherein the composition inhibits tumor growth with a lung cancer xenograft mouse model (A549 cell) better than GemZar.

48. The composition of claim 24, wherein the composition inhibits tumor growth with a pancreatic cancer xenograft mouse model (PANC-1 cell) better than GemZar.

49. A pharmaceutical composition comprising GEM-TAC and STP302.

50. A pharmaceutical composition comprising an siRNA oligo against human PDL-1 gene expression in combination with GEM-TAC.

51. A pharmaceutical composition comprising an siRNA oligo against human PDL-2 gene expression in combination with GEM-TAC.

52. A method of treating cancer in a human or inhibiting the growth of neoplastic or tumor cells in a human comprising the step of administering a therapeutically effective amount of the composition of any one of claims 47-51 to the human.

53. The method of claim 52, wherein the cancer is pancreatic cancer.
